SEQUENCE LISTING (4 sequences)

SEQ ID NO: 1 - DNA, 1796 bp, Homo sapiens
catcagacgggaagcctggactgtgggttgggggcagcctcagcctctccaacctggcacccactgcccgtggcccttaggcacctgcttggggtcctggagccccttaaggccaccagcaaatcctaggagaccgagtcttggcacgtgaacagagccagatttcacactgagcagctgcagtcggagaaatcagagaaagcgtcacccagccccagattccgaggggcctgccagggactctctcctcctgctccttggaaaggaagaccccgaaagacccccaagccaccggctcagacctgcttctgggctgccatgggacttgcggccaccgccccccggctgtcctccacgctgccgggcagataagggcagctgctgcccttggggcacctgctcactcccgcagcccagccactcctccagggccagcccttccctgactgagtgaccacctctgctgccccgaggccatgtaggccgtgcttaggcctctgtggacacactgctggggacggcgcctgagctctcagggggacgaggaacaccaccatgccccggggcttcacctggctgcgctatcttgggatcttccttggcgtggccttggggaatgagcctttggagatgtggcccttgacgcagaatgaggagtgcactgtcacgggttttctgcgggacaagctgcagtacaggagccgacttcagtacatgaaacactacttccccatcaactacaagatcagtgtgccttacgagggggtgttcagaatcgccaacgtcaccaggctgcagagggcccaggtgagcgagcgggagctgcggtatctgtgggtcttggtgagcctcagtgccactgagtcggtgcaggacgtgctgctcgagggccacccatcctggaagtacctgcaggaggtggagacgctgctgctgaatgtccagcagggcctcacggatgtggaggtcagccccaaggtggaatccgtgttgtccctcttgaatgccccagggccaaacctgaagctggtgcggcccaaagccctgctggacaactgcttccgggtcatggagctgctgtactgctcctgctgtaaacaaagctccgtcctaaactggcaggactgtgaggtgccaagtcctcagtcttgcagcccagagccctcattgcagtatgcggccacccagctgtaccctccgcccccgtggtcccccagctccccgcctcactccacgggctcggtgaggccggtcagggcacagggcgagggcctcttgccctgagcaccctggatggtgactgcggataggggcagccagaccagctcccacaggagttcaactgggtctgagacttcaaggggtggtggtgggagccccccttgggagaggacccctgggaagggtgtttttcctttgagggggattctgtgccacagcagggctcagcttcctgccttccatagctgtcatggcctcacctggagcggaggggacctggggacctgaaggtggatggggacacagctcctggcttctcctggtgctgccctcactgtccccccgcctaaagggggtactgagcctcctgtggcccgcagcagtgagggcacagctgtgggttgcaggggagacagccagcacggcgtggccattctatgaccccccagcctggcagactggggagctgggggcagagggcggtgccaagtgccacatcttgccatagtggatgctcttccagtttcttttttctattaaacaccccacttcctttggaaaaaaaaaaaaaaaaa

SEQ ID NO: 2 - protein, 242 aa, Homo sapiens
MPRGFTWLRYLGIFLGVALGNEPLEMWPLTQNEECTVTGFLRDKLQYRSRLQYMKHYFPINYKISVPYEGVFRIANVTRLQRAQVSERELRYLWVLVSLSATESVQDVLLEGHPSWKYLQEVETLLLNVQQGLTDVEVSPKVESVLSLLNAPGPNLKLVRPKALLDNCFRVMELLYCSCCKQSSVLNWQDCEVPSPQSCSPEPSLQYAATQLYPPPPWSPSSPPHSTGSVRPVRAQGEGLLP

SEQ ID NO: 3 - protein, 972 aa, Homo sapiens
MGPGVLLLLLVATAWHGQGIPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTPVVVACMSIMALLLLLLLLLLYKYKQKPKYQVRWKIIESYEGNSYTFIDPTQLPYNEKWEFPRNNLQFGKTLGAGAFGKVVEATAFGLGKEDAVLKVAVKMLKSTAHADEKEALMSELKIMSHLGQHENIVNLLGACTHGGPVLVITEYCCYGDLLNFLRRKAEAMLGPSLSPGQDPEGGVDYKNIHLEKKYVRRDSGFSSQGVDTYVEMRPVSTSSNDSFSEQDLDKEDGRPLELRDLLHFSSQVAQGMAFLASKNCIHRDVAARNVLLTNGHVAKIGDFGLARDIMNDSNYIVKGNARLPVKWMAPESIFDCVYTVQSDVWSYGILLWEIFSLGLNPYPGILVNSKFYKLVKDGYQMAQPAFAPKNIYSIMQACWALEPTHRPTFQQICSFLQEQAQEDRRERDYTNLPSSSRSGGSGSSSSELEEESSSEHLTCCEQGDIAQPLLQPNNYQFC

SEQ ID NO: 4 - protein, 498 aa, Homo sapiens
IPVIEPSVPELVVKPGATVTLRCVGNGSVEWDGPPSPHWTLYSDGSSSILSTNNATFQNTGTYRCTEPGDPLGGSAAIHLYVKDPARPWNVLAQEVVVFEDQDALLPCLLTDPVLEAGVSLVRVRGRPLMRHTNYSFSPWHGFTIHRAKFIQSQDYQCSALMGGRKVMSISIRLKVQKVIPGPPALTLVPAELVRIRGEAAQIVCSASSVDVNFDVFLQHNNTKLAIPQQSDFHNNRYQKVLTLNLDQVDFQHAGNYSCVASNVQGKHSTSMFFRVVESAYLNLSSEQNLIQEVTVGEGLNLKVMVEAYPGLQGFNWTYLGPFSDHQPEPKLANATTKDTYRHTFTLSLPRLKPSEAGRYSFLARNPGGWRALTFELTLRYPPEVSVIWTFINGSGTLLCAASGYPQPNVTWLQCSGHTDRCDEAQVLQVWDDPYPEVLSQEPFHKVTVQSLLTVETLEHNQTYECRAHNSVGSGSWAFIPISAGAHTHPPDEFLFTP

(19) United States
(12) Patent Application Publication
(10) Pub. No.: US 2018/0113139 A1
(43) Pub. Date: Apr. 26, 2018

(54) METHODS AND COMPOSITIONS FOR DIAGNOSING AND TREATING INFLAMMATORY BOWEL DISORDERS

(71) Applicant: Nogra Pharma Limited, Dublin 2 (IE)

(72) Inventors: Salvatore Bellinvia, Mendrisio (CH); Francesca Viti, Salorino (CH)

(21) Appl. No.: 15/329,514

(22) PCT Filed: Jul. 28, 2015
(86) PCT No.: PCT/EP2015/067306

§ 371 Date: Jan. 26, 2017

Related U.S. Application Data
(60) Provisional application No. 62/029,875, filed on Jul. 28, 2014.

(51) Int. Cl. G01N 33/68; C12Q 1/6883

(52) U.S. Cl. CPC G01N 33/6869; C12Q 1/6883; G01N 2333/54; C12Q 2600/106; G01N 2800/52; C12Q 2600/158; G01N 2800/065

(57) ABSTRACT

Disclosed herein are methods for diagnosing and/or treating inflammatory bowel diseases associated with elevated activity or expression of IL-34. Also disclosed are pharmaceutical compositions containing an inhibitor of IL-34 useful for treating inflammatory bowel diseases and manufacture of medicaments containing an inhibitor of IL-34 to be used in treating inflammatory bowel disease.

### FIELD OF THE INVENTION

The present invention is generally directed towards methods of treating inflammatory bowel diseases associated with inflammatory cytokine production via administration of inhibitors of IL-34, as well as pharmaceutical compositions containing inhibitors of IL-34 for use in treating inflammatory bowel diseases. The invention also discloses methods of diagnosing inflammatory bowel diseases by detecting IL-34 expression signal.

### BACKGROUND

Inflammatory bowel disease is a chronic inflammatory disorder of the gastrointestinal tract suffered by approximately 1.4 million patients in the United States. It is one of the five most prevalent gastrointestinal disease burdens in the United States, with an overall health care cost of more than $1.7 billion. Each year in the United States, inflammatory bowel disease accounts for more than 700,000 physician visits, 100,000 hospitalizations, and disability in 119,000 patients. No medical cure currently exists, so disease management requires a lifetime of care.

The two most common forms of inflammatory bowel disease are Crohn's disease and ulcerative colitis. Although Crohn's disease can affect the entire gastrointestinal tract, it primarily affects the ileum (the distal or lower portion of the small intestine) and the large intestine. Ulcerative colitis primarily affects the colon and the rectum. The etiology of inflammatory bowel disease is not completely understood, although both environmental and genetic factors are believed to play a role in the disease. Environmental components may include alterations in flora of the gut which are affected by exposure to ingested foods and medications.

Inflammatory bowel disease is associated with abdominal pain, vomiting, diarrhea, rectal bleeding, severe cramps, muscle spasms, weight loss, malnutrition, fever, and anemia. Patients with inflammatory bowel disease may also suffer from skin lesions, joint pain, eye inflammation, and liver disorders, and children suffering from ulcerative colitis may suffer from growth defects. Less commonly, patients may suffer from arthritis, pyoderma gangrenosum, primary sclerosing cholangitis, and non-thyroidal illness syndrome. Although rarely fatal, these symptoms decrease quality of life for patients. However, in some cases complications of inflammatory bowel disease, including toxic megacolon, bowel perforation, and surgical complications can result in death. Additionally, patients have an increased risk of colorectal cancer, and may be more likely to suffer endothelial dysfunction and coronary artery disease.

Diagnostic criteria for inflammatory bowel disease are not well established due to a lack of knowledge of disease etiology. Additionally, patient response to available therapies varies with disease severity and can vary over cycles of active inflammation and remission. Moreover, many of the current therapies for inflammatory bowel disease are associated with undesirable side effects and fail to provide relief for a large number of patients. For instance, approximately three quarters of patients with Crohn's disease require surgery because available medications ultimately cannot provide relief from disease symptoms. However, even after surgery, symptoms of Crohn's may recur. Similarly, approximately one-third of patients with ulcerative colitis undergo colectomy because available drugs fail to provide relief.

Thus, there is a pressing need to identify reliable methods of diagnosing inflammatory bowel disease. There is a further need to identify methods of treatment that provide effective and permanent relief from symptoms across a broad spectrum of patients and which are not associated with negative side effects or cycles of inflammation and remission. The need to identify new therapeutic options is also necessary in order to provide alternatives to the extreme, and sometimes ineffective, surgical remedies that many patients with inflammatory bowel disease face.

### SUMMARY

The invention described herein provides novel methods and compositions for treating and/or diagnosing inflammatory bowel disease. The invention takes advantage of the discoveries that IL-34 expression is elevated in inflammatory bowel disease and that IL-34 expression promotes increased expression of other inflammatory cytokines known to play a role in inflammatory bowel disease. While other diagnostic methods for evaluating inflammatory bowel disease have been proposed, the present invention provides new methods of diagnosing patients with inflammatory bowel disease and determining the suitability of patients for treatment of inflammatory bowel disease using an IL-34 inhibitor. Similarly, while other potential targets for therapeutic intervention in inflammatory bowel disease have been proposed, the present invention provides new compositions and methods of treating inflammatory bowel disease by inhibiting a novel target, i.e. IL-34.

The present invention provides a method for treating an inflammatory bowel disease, e.g. Crohn's disease or ulcerative colitis, by inhibiting IL-34. Inhibiting IL-34 may be accomplished by preventing or reducing expression of IL-34 gene products, e.g. IL-34 protein or IL-34 RNA. Inhibiting IL-34 may also be accomplished by inhibiting IL-34 signaling activity (e.g., by sequestering IL-34 protein or RNA), sequestering IL-34 binding partners or receptors (e.g., Colony Stimulating Factor 1 Receptor (CSF1R), also known as Macrophage Colony Stimulating Factor Receptor 1 (M-CSFR-1)), inhibiting the activity of proteins activated by IL-34 signaling, or binding to a specific moiety or moieties of IL-34 necessary for its functional interaction with other cellular components. The invention specifically provides methods benefiting from inhibiting IL-34 in a patient.

In one embodiment the invention provides a method of treating inflammatory bowel disease by administering to a patient in need thereof an effective amount of an inhibitor of IL-34. An inhibitor of IL-34 may be any reagent capable of inhibiting IL-34 expression or activity in a targeted manner For example, inhibitors of IL-34 include, but are not limited to, antisense IL-34 therapies (i.e., antisense oligonucleotides against IL-34), antibodies (e.g., antibodies that bind IL-34, IL-34 binding partners or IL-34 receptors), peptide inhibitors (e.g., peptides that include all or a portion of the CSF1R extracellular domain), and small molecules (e.g., small molecules that bind or inhibit IL-34, IL-34 gene products, IL-34 binding partners, or IL-34 receptors).

An antisense oligonucleotide against IL-34 may range from, for example, 8 to 40 nucleotides in length, and target an IL-34 RNA transcript for degradation, impaired transcript processing, or impaired protein translation. An antisense oligonucleotide against IL-34 may target the IL-34 nucleotide sequence (NCBI Reference Sequence NM_152456.2; SEQ ID NO: 1). An inhibitor of IL-34 may be an antibody, such as, for example, a full-length antibody, a chimeric antibody, a Fab′, a Fab, a F(ab′)2, a single domain antibody (DAB), an Fv, a single chain Fv (scFv), a minibody, a diabody, a triabody, or a mixture thereof. An antibody inhibitor of IL-34 may be an antibody directed against, for example, IL-34 protein sequence (NCBI Reference Sequence NP_689669.2; SEQ ID NO: 2) or CSF1R protein sequence (NCBI Reference Sequence: NP_005202.2; SEQ ID NO: 3). An inhibitor of IL-34 may be a peptide inhibitor that includes all or a portion of an extracellular domain of Colony Stimulating Factor 1 Receptor, for example, residues 20-517 of NCBI Reference Sequence: NP_005202.2 (SEQ ID NO: 4). An inhibitor of IL-34 may be a small molecule capable of binding to IL-34 or an IL-34 interaction partner, for example, an IL-34 interaction protein, for example CSF1R.

An inhibitor of IL-34 may include an inhibitor of the IL-34 gene or gene products, i.e. IL-34 RNA or IL-34 protein, or any of the cellular constituents with which the IL-34 gene or gene products interact directly. For instance, an inhibitor of IL-34 may be, but is not limited to, an antisense oligonucleotide against IL-34 or an IL-34 interaction partner, an antibody, peptide, or small molecule that sequesters IL-34 or an IL-34 interaction partner, or an antibody, peptide, or small molecule that binds to IL-34 or an IL-34 interaction partner in a manner that prevents a direct interaction between IL-34 and other interaction partners critical for IL-34 signaling. An IL-34 interaction partner may be a protein, nucleotide, or any other cellular or extracellular component with which IL-34 interacts directly to promote IL-34 signaling. In addition, an IL-34 inhibitor may include an inhibitor of any interaction partner with which IL-34 interacts in order to promote IL-34 signaling. In some instances, an IL-34 interaction partner may be part of a protein complex with which IL-34 directly interacts. In one embodiment of the invention, an inhibitor of IL-34 may be an inhibitor of a receptor of IL-34, e.g. CSF1R.

The invention provides a method of inhibiting inflammatory cytokine production by inhibiting IL-34 in cells of a patient suffering from an inflammatory bowel disease. The invention also provides a method of inhibiting inflammatory cytokine production in cells of a patient suffering from an inflammatory bowel disease by administering an effective amount of an inhibitor of IL-34. Inflammatory cytokines include cytokines that help initiate an inflammatory response in cells characterized by recruitment of granulocytes and lymphocytes. Examples of inflammatory cytokines include, but are not limited to, IL-1, IL-6, IL-8, and TNF-alpha. Production of inflammatory cytokines (e.g., IL-6, IL-8, and TNF-alpha) is increased as a result of increased IL-34 expression or increased IL-34 signaling. The present invention leverages this unexpected discovery to provide a means of inhibiting inflammatory cytokine production through the inhibition of IL-34 expression and/or activity in cells of a patient with inflammatory bowel disease.

Thus, the invention also provides a method of inhibiting inflammatory cytokine production in cells of a patient suffering from an inflammatory bowel disease, wherein such inhibition reduces or inhibits an IL-34 mediated inflammatory response. The invention also provides a method of reducing or inhibiting an IL-34 mediated inflammatory response in cells of a patient suffering from an inflammatory bowel disease by administering an effective amount of an inhibitor of IL-34. An inflammatory response may be characterized by expression of inflammatory cytokines, movement of plasma and leukocytes from blood to tissue in which the inflammatory signal is detected by the immune system, and swelling or inflammation of the affected tissue.

Inhibiting IL-34 may occur in any cell in which IL-34 may be expressed or activated, or in any cell in which IL-34 signaling may be activated, increased, or altered. For instance IL-34 may be inhibited in a cell that expresses IL-34 including, but not limited to, cells of the gastrointestinal tract, such as colonic mucosal cells or ileal mucosal cells, for instance, colonic or ileal lamina propria mononuclear cells. Additionally, inhibiting IL-34 may occur by inhibiting interaction of IL-34 with a cell that expresses an IL-34 receptor (e.g., CSF1R) such as a bone marrow cell, a monocyte, or a macrophage.

The invention also provides a method of treating an inflammatory bowel disease associated with altered IL-34 expression in a patient in need thereof, by administering an effective amount of an inhibitor of IL-34. In general, an inflammatory bowel disease associated with altered IL-34 expression correlates with increased IL-34 expression. Thus, an inhibitor of IL-34 is useful to reduce levels of IL-34 expression or activity in a patient suffering from an inflammatory bowel disease. In general, administering an effective amount of an IL-34 inhibitor reduces IL-34 activity or expression to lower levels than those associated with the active inflammatory bowel disease state.

The invention also provides a method of treating inflammatory bowel disease via administration of a pharmaceutically acceptable formulation of an inhibitor of IL-34. In certain embodiments, the invention provides a method of treating inflammatory bowel disease via administration of a pharmaceutically acceptable formulation of an inhibitor of IL-34 administered to a patient in need thereof. Pharmaceutically acceptable formulations of inhibitors of IL-34 include formulations known in the art to be safe for administration and tolerated by animals, especially humans. Such formulations should also be efficacious in terms of delivering any active agent or agents included in the formulation to the intended site of disease treatment. A patient in need of administration of a pharmaceutically acceptable formulation of an inhibitor of IL-34 includes a patient suffering from inflammatory bowel disease (e.g., a patient diagnosed with inflammatory bowel disease), a patient at risk for development of inflammatory bowel disease (e.g., a patient diagnosed as having a high risk of developing inflammatory bowel disease), a patient being treated prophylactically for inflammatory bowel disease, a patient currently being treated for inflammatory bowel disease, or a patient in which inflammatory bowel disease has been effectively treated, ameliorated, or reduced in severity, including a patient in which inflammatory bowel disease has been ameliorated or a patient in which inflammatory bowel disease had been ameliorated but has returned.

The present invention also provides a pharmaceutical composition for use in treating inflammatory bowel disease that may include an inhibitor of IL-34 and a pharmaceutically acceptable carrier. In some embodiments of the invention, for instance, a method of treating inflammatory bowel disease, a method of inhibiting inflammatory cytokine production in cells of a patient suffering from inflammatory bowel disease, a method of reducing or inhibiting an IL-34 mediated inflammatory response in cells of a patient suffering from an inflammatory bowel disease, or a method of treating inflammatory bowel disease associated with altered IL-34 expression in a patient in need thereof may include administering a pharmaceutical composition comprising an inhibitor of IL-34 and a pharmaceutically acceptable carrier. Additionally, the invention provides a use of an inhibitor of IL-34 in the manufacture of a medicament for the treatment of an inflammatory bowel disease.

In one embodiment the present invention provides an inhibitor of IL-34 for use in the treatment of inflammatory bowel disease.

The present invention also provides an inhibitor of IL-34 for use in inhibiting inflammatory cytokine production, in a patient suffering from an inflammatory bowel disease.

The inhibitor of IL-34 for use in inhibiting inflammatory cytokine production, reduces or inhibits an IL-34 mediated inflammatory response, in cells of a patient suffering from an inflammatory bowel disease.

The inhibitor of IL-34 for use in the treatment of inflammatory bowel disease, reduces or inhibits an IL-34 mediated inflammatory response in cells of a patient suffering from an inflammatory bowel disease.

Suitably, the inhibitor of IL-34 for use in treating inflammatory bowel disease, is formulated as a pharmaceutically acceptable formulation of an IL-34 inhibitor.

The pharmaceutically acceptable formulation of an IL-34 inhibitor for use in treating inflammatory bowel disease is for administration to a patient in need thereof.

The present invention provides an IL-34 inhibitor for use in reducing or inhibiting an IL-34 mediated inflammatory response in cells of a patient suffering from an inflammatory bowel disease.

The present invention also provides an inhibitor of IL-34 for use in the treatment of inflammatory bowel disease associated with altered IL-34 expression.

The IL-34 inhibitor for use in the treatment of inflammatory bowel disease may be an inhibitor of the IL-34 receptor. The receptor of IL-34 may be Colony Stimulating Factor 1 Receptor.

The inhibitor of IL-34 for use in the treatment of inflammatory bowel disease may comprise an antisense oligonucleotide against IL-34. The antisense oligonucleotide may be from 8 to 40 nucleotides in length.

The inhibitor of IL-34 for use in the treatment of inflammatory bowel disease may be an antibody. The antibody for use in the treatment of inflammatory bowel disease may be selected from the group consisting of a full-length antibody, a chimeric antibody, a Fab′, a Fab, a F(ab′)2, a single domain antibody (DAB), an Fv, a single chain Fv (scFv), a minibody, a diabody, a triabody, or a mixture thereof.

The antibody for use in the treatment of inflammatory bowel disease may be an antibody directed against the IL-34 protein sequence (SEQ ID NO: 2) or Colony Stimulating Factor 1 Receptor protein sequence (SEQ ID NO: 3).

In another embodiment, the inhibitor of IL-34 for use in the treatment of inflammatory bowel disease may be a peptide inhibitor.

The peptide inhibitor for use in the treatment of inflammatory bowel disease may comprise a portion of an extracellular domain of Colony Stimulating Factor 1 Receptor (SEQ ID NO: 4).

The inhibitor of IL-34 for use in the treatment of inflammatory bowel disease according to the present invention may be for administration topically, parenterally, orally, pulmonarily, intratracheally, intranasally, transdermally, or intraduodenally.

The present invention also provides a pharmaceutical composition for use in treating inflammatory bowel disease, comprising an inhibitor of IL-34 and a pharmaceutically acceptable carrier. For example, the pharmaceutical composition for use in treating inflammatory bowel disease, according to the present invention may comprise an antisense oligonucleotide against IL-34 and a pharmaceutically acceptable carrier. The antisense oligonucleotide may be from 8 to 40 nucleotides in length.

The pharmaceutical composition for use in treating inflammatory bowel disease may comprise an inhibitor of IL-34 and a pharmaceutically acceptable carrier, and said inhibitor of IL-34 may be an antibody. The antibody may be selected from the group consisting of a full-length antibody, a chimeric antibody, a Fab′, a Fab, a F(ab′)2, a single domain antibody (DAB), an Fv, a single chain Fv (scFv), a minibody, a diabody, a triabody, or a mixture thereof.

The pharmaceutical composition for use in treating inflammatory bowel disease may comprise an antibody directed against the IL-34 protein sequence (SEQ ID NO: 2) or Colony Stimulating Factor 1 Receptor protein sequence (SEQ ID NO: 3) and a pharmaceutically acceptable carrier.

In another embodiment, the pharmaceutical composition for use in treating inflammatory bowel disease may comprise an inhibitor of IL-34 and a pharmaceutically acceptable carrier, wherein said inhibitor is a peptide inhibitor. The peptide inhibitor may comprise a portion of an extracellular domain of Colony Stimulating Factor 1 Receptor (SEQ ID NO: 4).

The pharmaceutical composition for use in treating inflammatory bowel disease according to the present invention may be for administration topically, parenterally, orally, pulmonarily, intratracheally, intranasally, transdermally, or intraduodenally.

The inhibitor of IL-34 for use in treating inflammatory bowel disease according to the present invention, may for example be used in treating Crohn's disease, gastroduodenal Crohn's disease, Crohn's (granulomatous) colitis, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, and indeterminate colitis.

The pharmaceutical composition for use in treating inflammatory bowel disease according to the present invention, for example, may be used in treating Crohn's disease, gastroduodenal Crohn's disease, Crohn's (granulomatous) colitis, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, and indeterminate colitis.

Methods of the invention include methods of administering an inhibitor of IL-34 or a pharmaceutical composition that includes an inhibitor of IL-34 and a pharmaceutically acceptable carrier. According to the invention, an inhibitor of IL-34 may be administered topically, parenterally, orally, pulmonarily, intratracheally, intranasally, transdermally, or intraduodenally. Similarly, a pharmaceutical composition that includes an inhibitor of IL-34 and a pharmaceutically acceptable carrier may be administered topically, parenterally, orally, pulmonarily, intratracheally, intranasally, transdermally, or intraduodenally. The route of administration may depend upon the method being employed, the disease state, and the expertise of the individual administering treatment (e.g., a physician), as well as other factors.

The invention also provides diagnostic methods useful for diagnosing a patient with inflammatory bowel disease through evaluation of IL-34 expression signal. In one embodiment the invention provides a method of diagnosing a patient with inflammatory bowel disease by detecting levels of IL-34 expression signal in one or more biological samples of a patient. In another embodiment, the invention provides a method of determining whether a patient suffering from inflammatory bowel disease is a candidate for treatment with an inhibitor of IL-34 by detecting levels of IL-34 expression signal in one or more biological samples of the patient. In embodiments of the invention, the IL-34 expression signal may be a peptide, protein, or RNA signal. IL-34 expression signal can include any indication of IL-34 gene expression, or gene product activity. Indices of IL-34 gene expression that can be assessed include, but are not limited to, IL-34 gene or chromatin state, IL-34 gene interaction with cellular components that regulate gene expression, IL-34 gene product expression levels (e.g., IL-34 RNA expression levels, IL-34 protein expression levels), or interaction of IL-34 RNA or protein with transcriptional, translational, or post-translational processing machinery. Indices of IL-34 gene product activity include, but are not limited to, assessment of IL-34 signaling activity (e.g., assessment of CSF1R activation or MAPK1/MAPK3 phosphorylation) and assessment of IL-34 receptor binding (e.g., CSF1R binding). Detection of IL-34 expression signal may be accomplished through in vivo, in vitro, or ex vivo methods. In a preferred embodiment, methods of the invention may be carried out in vitro.

In embodiments of the invention, a biological sample may be a blood sample, a tissue sample, a cell sample, a serum sample, or a fecal matter sample. In general, a biological sample may be any sample taken from a patient or a suitable control patient (i.e., an individual not suffering from inflammatory bowel disease) that is useful for assaying the absolute level or relative level of IL-34 expression or activity in a patient. In a preferred embodiment, the biological sample is taken from the gastrointestinal tract of the patient.

In certain embodiments of the invention, IL-34 expression signal is compared to IL-34 expression signal from anatomically equivalent biological samples of one or more patients not suffering from inflammatory bowel disease, anatomically equivalent biological samples of one or more patients suffering from inflammatory bowel disease, and/or anatomically equivalent biological samples of one or more patients treated for inflammatory bowel disease. Anatomically equivalent biological samples are samples of cells, tissue, serum, or blood taken from the same patient, the same organ, the identical organ in a different patient, or generally from the same location in the body of the same or a different patient. For instance, anatomically equivalent biological samples may be taken from a specific site in the gastrointestinal tract (e.g., the ileum, colon, or rectum) of two different patients (i.e., a patient to be diagnosed with inflammatory bowel disease or a patient suffering from inflammatory bowel disease and a patient not suffering from inflammatory bowel disease, another patient suffering from inflammatory bowel disease, or a patient treated for inflammatory bowel disease). Methods of the invention can also be used to monitor the efficacy of treating a patient suffering from inflammatory bowel disease by assessing the effect of treatment on IL-34 expression signal. Hence, in one embodiment of the invention, the anatomically equivalent biological samples come from the same patient before and after treatment with an inhibitor of IL-34. In another embodiment of the invention, the patient is receiving at least one inhibitor of IL-34 when at least one biological sample is obtained.

In certain embodiments of the invention, a patient suffering from inflammatory bowel disease is treated. A patient may be any animal suffering from inflammatory bowel disease, preferably a mammal, preferably a human. In various embodiments of the invention, the inflammatory bowel disease may be any of the following: Crohn's disease, gastroduodenal Crohn's disease, Crohn's (granulomatous) colitis, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, and indeterminate colitis. In a preferred embodiment of the invention, the inflammatory bowel disease is Crohn's disease. In another preferred embodiment of the invention, the inflammatory bowel disease is ulcerative colitis.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A shows a bar graph of IL-34 mRNA expression in colonic biopsies from control patients (Colonic CTR), patients with ulcerative colitis (UC), and patients with Crohn's disease (Colonic CD); FIG. 1B shows a bar graph of IL-34 mRNA expression in ileal biopsies from control patients (Ileal CTR) and patients with Crohn's disease (Ileal CD); FIG. 1C shows a bar graph of IL-34 mRNA expression in colonic biopsies from control patients (CTR), inflammatory bowel disease patients receiving no therapy (w/o therapy), or inflammatory bowel disease patients receiving either mesalamine (5-ASA), steroids (CS), or immunomodulators (ISS); and FIG. 1D shows a graph of IL-34 mRNA expression in mucosa taken from involved (Involv) or uninvolved (Uninv) regions of 9 individual inflammatory bowel disease patients' mucosa.

FIG. 2A is a Western blot showing IL-34 (upper panel) and β-actin (lower panel) expression signal in total protein extract from colonic mucosal cells of 3 control patients (CTR) and 3 patients each with ulcerative colitis (UC) or Crohn's disease (CD); FIG. 2B is a Western blot showing IL-34 (upper panel) and β-actin (lower panel) expression signal in total protein extract from ileal mucosal cells of 3 control patients (Ileal CTR) and 3 patients with Crohn's disease (Ileal CD); FIG. 2C shows photomicrographs (left) taken at 100× magnification of colonic tissue sections stained with either control IgG (IgG) or IL-34 antibody taken from control (CTR), ulcerative colitis (UC), or Crohn's disease (CD) patients, and a bar graph (right panel) of the number of IL-34 positive cells per high power field (IL-34⁺ cells/hpf) in IL-34 stained CTR, UC, or CD colonic tissue sections; and FIG. 2D shows photomicrographs (left) taken at 100× magnification of ileal tissue sections stained with IL-34 antibody taken from control (Ileal CTR), or Crohn's disease (Ileal CD) patients, and a bar graph (right panel) of the number of IL-34 positive cells per high power field (IL-34⁺cells/hpf) in IL-34 stained Ileal CTR and Ileal CD tissue sections.

FIG. 3A is a bar graph showing expression of CSF1R mRNA in colonic biopsies taken from 9 controls (Colonic CTR), 14 UC patients, and 8 CD patients, analyzed by real-time PCR. CSF1R levels were normalized to β-actin levels. Data are expressed as mean±SEM. FIG. 3B shows representative photomicrographs (100× original magnification) of isotype control (IgG) or CSF1R antibody stained paraffin-embedded colonic tissue sections from 1 control (CTR) patient, 1 patient with CD, and 1 patient with UC.

FIG. 4A is a bar graph showing CSF1R mRNA expression in DLD-1, HCT-116, HT-29, and NCM-460 evaluated by real-time PCR, with expression levels normalized to β-actin expression levels. FIG. 4B is a representative Western blot showing CSF1R and β-actin signal in total protein extracts taken from DLD-1, HCT-116, NCM-460 and HT-29 cells.

FIG. 5A shows a bar graph of IL-34 mRNA expression in colonic mucosal explants as measured by real-time PCR following 6 hours without stimulation (UNST) or in the presence of TNF-alpha at 20 ng/ml (TNF-α), IL-6 at 50 ng/ml (IL-6), or Interferon-gamma at 100 ng/ml (IFN-γ); FIG. 5B shows a graph of IL-34 RNA expression levels in colonic tissue biopsies taken from individual IBD patients before (PRE) and after (POST) infliximab (Remicade®) treatment as measured by real-time PCR; FIG. 5C shows a graph of IL-34 mRNA expression in colonic mucosal explants from inflammatory bowel disease patients (IBD Explants) exposed to either control IgG (IgG) or the TNF-alpha inhibitor infliximab (Remicade®, (IFX)) for 24 hours; and FIG. 5D shows a graph of IL-34 mRNA expression in colonic lamina propria nuclear cells harvested from inflammatory bowel disease patients (IBD LPMC) exposed to either control IgG (IgG) or infliximab (Remicade®, (IFX)) for 18 hours.

FIG. 6 shows a series of bar graphs that display observed levels of TNF-alpha (FIG. 4A, TNF-a), IL-8 (FIG. 4B, IL-8), or IL-6 (FIG. 4C, IL-6) mRNA expression in normal colonic lamina propria mononuclear cells following 6 hours of exposure to either no stimulation (Unst) or 25 ng/ml, 50 ng/ml, or 100 ng/ml of recombinant human IL-34.

FIG. 7, panels A-I, show mRNA expression of individual chemokines in serum-starved DLD-1 cells which were either left unstimulated (Unst) or stimulated with 50 ng/ml of human recombinant IL-34 for 1.5 hours, 3 hours, or 6 hours. CCL2 (FIG. 7A), CCL3 (FIG. 7B), CCLS (FIG. 7C), CCL17 (FIG. 7D), CCL20 (FIG. 7E), CCL24 (FIG. 7F), CCL25 (FIG. 7G), CXCL9 (FIG. 7H), and CXCL10 (FIG. 71) RNA transcripts were analyzed by real-time PCR, and expression levels were normalized to β-actin mRNA levels. Data for 6 independent experiments is expressed as the mean±SEM. FIG. 7J is a bar graph showing CCL20 mRNA expression in DLD-1 cells left unstimulated or cultured in the presence of 25 ng/ml, 50 ng/ml, or 100 ng/ml IL-34 for 3 hours. CCL20 mRNA transcript expression was analyzed by real-time PCR, and expression levels were normalized to β-actin mRNA levels. Data for 6 independent experiments is expressed as the mean±SEM.

FIG. 8A shows a Western blot of total individual MAP kinases (ERK1/2, p38, JNK) and phosphorylated MAP kinase protein fractions (p-ERK1/2, p-p38, p-JNK) in extracts of serum-starved DLD-1 cells left untreated (Unst) or treated with IL-34 at 50 ng/ml for 5 minutes (5′), 10 minutes (10′), 15 minutes (15′), 30 minutes (30′), or 60 minutes (60′). FIG. 8B is a bar graph showing CCL20 protein expression evaluated by ELISA in cell-free supernatants from serum-starved DLD-1 cells pre-incubated with specific inhibitors of ERK1/2 (PD98059) or p38 (SB202190) or dimethyl sulfoxide (Dmso) for 1 hour and then stimulated with IL-34 at 50 ng/mL for 48 hours. Data for 6 independent experiments is expressed as the mean±SEM.

### DETAILED DESCRIPTION

### Inflammatory Bowel Disease

The present invention provides methods for treatment of inflammatory bowel disease. “Inflammatory bowel disease,” as used herein, refers to a number of chronic inflammatory diseases including Crohn's disease, gastroduodenal Crohn's disease, Crohn's (granulomatous) colitis, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, and indeterminate colitis. Crohn's disease and ulcerative colitis are the two most common forms of inflammatory bowel disease. Inflammatory bowel disease is an autoimmune disease of the digestive system. Crohn's disease may be localized to any portion of the gastrointestinal tract, including the terminal ileum, and may impact all cell types of the gastrointestinal tract. Ulcerative colitis is localized to the colon and rectum, and affects cells of the mucosa only.

Both environmental and genetic factors are believed to play a role in inflammatory bowel disease, although the identity of such factors is not well-defined. Environmental components may include alterations in flora of the gut which are affected by exposure to ingested foods and medications.

Inflammatory bowel disease is associated with symptoms including abdominal pain, vomiting, diarrhea, rectal bleeding, severe cramps, muscle spasms, weight loss, malnutrition, fever, anemia, skin lesions, joint pain, eye inflammation, liver disorders, arthritis, pyoderma gangrenosum, primary sclerosing cholangitis, and non-thyroidal illness syndrome. Children suffering from ulcerative colitis may suffer from growth defects.

### Treatment and Evaluation

A “patient,” as described herein, refers to any animal at risk for, suffering from or diagnosed for inflammatory bowel disease, including, but not limited to, mammals, primates, and humans. In certain embodiments, the patient may be a non-human mammal such as, for example, a cat, a dog, or a horse. A patient may be an individual diagnosed with a high risk of developing inflammatory bowel disease, someone who has been diagnosed with inflammatory bowel disease, someone who previously suffered from inflammatory bowel disease, or an individual evaluated for symptoms or indications of inflammatory bowel disease, for example, IL-34 expression signal.

“A patient in need,” as used herein, refers to a patient suffering from any of the symptoms or manifestations of inflammatory bowel disease, a patient who may suffer from any of the symptoms or manifestations of inflammatory bowel disease, or any patient who might benefit from a method of the invention for treating inflammatory bowel disease. A patient in need may include a patient who is diagnosed with a risk of developing inflammatory bowel disease, a patient who has suffered from inflammatory bowel disease in the past, or a patient who has previously been treated for inflammatory bowel disease. Of particular relevance are individuals that suffer from inflammatory bowel disease associated with increased levels of IL-34 expression or activity.

The terms “treat”, “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it; (b) inhibiting the disease, i.e. preventing the disease from increasing in severity or scope; (c) relieving the disease, i.e. causing partial or complete amelioration of the disease; or (d) preventing relapse of the disease, i.e. preventing the disease from returning to an active state following previous successful treatment of symptoms of the disease or treatment of the disease.

“Effective amount,” as used herein, refers to the amount of an agent that is sufficient to at least partially treat a condition when administered to a patient. The therapeutically effective amount will vary depending on the severity of the condition, the route of administration of the component, and the age, weight, etc. of the patient being treated. Accordingly, an effective amount of an inhibitor of IL-34 is the amount of inhibitor necessary to treat inflammatory bowel disease in a patient such that administration of the agent prevents inflammatory bowel disease from occurring in a subject, prevents inflammatory bowel disease progression (e.g., prevents the onset or increased severity of symptoms of inflammatory bowel disease such as rectal bleeding, anemia, or gastrointestinal inflammation), or relieves or completely ameliorates all associated symptoms of inflammatory bowel disease, i.e. causes regression of the disease.

Efficacy of treatment may be evaluated by means of evaluation of gross symptoms associated with inflammatory bowel disease, analysis of tissue histology, biochemical assay, imaging methods such as, for example, magnetic resonance imaging, or other known methods. For instance, efficacy of treatment may be evaluated by analyzing gross symptoms of the disease such as changes in abdominal pain, vomiting, diarrhea, rectal bleeding, cramps, muscle spasms, weight loss, malnutrition, fever, anemia or other aspects of gross pathology associated with inflammatory bowel disease following administration of an IL-34 inhibitor to a patient suffering from inflammatory bowel disease.

Efficacy of treatment may also be evaluated at the tissue or cellular level, for example, by means of obtaining a tissue biopsy (e.g., a gastrointestinal tissue biopsy) and evaluating gross tissue or cell morphology or staining properties. Biochemical assays that examine protein or RNA expression may also be used to evaluate efficacy of treatment. For instance, one may evaluate IL-34, IL-6, IL-8, TNF-alpha, or levels of another protein or gene product indicative of inflammatory bowel disease, inflammatory cytokine production, or an IL-34 mediated inflammatory response in dissociated cells or non-dissociated tissue via immunocytochemical, immunohistochemical, Western blotting, or Northern blotting methods, or methods useful for evaluating RNA levels such as quantitative or semi-quantitative polymerase chain reaction. One may also evaluate the presence or level of expression of useful biomarkers found in fecal matter, plasma, or serum to evaluate disease state and efficacy of treatment.

In evaluating efficacy of treatment, suitable controls may be chosen to ensure a valid assessment. For instance, one can compare symptoms evaluated in a patient with inflammatory bowel disease following administration of an inhibitor of IL-34 to those symptoms in the same patient prior to treatment or at an earlier point in the course of treatment or in another patient not diagnosed with inflammatory bowel disease. Alternatively, one may compare the results of biochemical or histological analysis of gastrointestinal tissue following administration of an IL-34 inhibitor with those of gastrointestinal tissue from the same patient or from an individual not diagnosed with inflammatory bowel disease or from the same patient prior to administration of the IL-34 inhibitor. Additionally, one may compare blood, serum, cell, or fecal samples following administration of an IL-34 inhibitor with comparable samples from an individual not diagnosed with inflammatory bowel disease or from the same patient prior to administration of the IL-34 inhibitor.

Validation of IL-34 inhibition may be determined by direct or indirect assessment of IL-34 expression levels or activity. For instance, biochemical assays that measure IL-34 protein or RNA expression may be used to evaluate overall IL-34 inhibition. For instance, one may measure IL-34 protein levels in gastrointestinal tissue by Western blot to evaluate overall IL-34 levels. One may also measure IL-34 mRNA levels by means of Northern blot or quantitative polymerase chain reaction to determine overall IL-34 inhibition. One may also evaluate IL-34 protein levels or levels of another protein indicative of IL-34 signaling activity in dissociated cells, non-dissociated tissue, blood, serum, or fecal matter via immunocytochemical or immunohistochemical methods.

IL-34 inhibition may also be evaluated indirectly by measuring parameters such as macrophage or monocyte generation or proliferation, or measuring alterations in other parameters correlated with changes in IL-34 activity, including MAP kinase phosphorylation and other indicators of CSF1R signaling activation. For instance, one may measure levels of active MAPK1 or MAPK3 phosphorylation in cells of a patient treated with an IL-34 inhibitor as an indication of IL-34 activity in said cells. One may also evaluate the presence or level of expression of useful biomarkers found in plasma, blood, fecal matter or tissue to evaluate efficacy of IL-34 inhibition.

Methods of treatment disclosed herein include methods of inhibiting inflammatory cytokine production. “Inflammatory cytokine production” refers to the expression of cytokines that initiate and/or promote an inflammatory cytokine response. An “inflammatory cytokine response” refers to an immune response that may be characterized by granulocyte recruitment, lymphocyte recruitment, systemic inflammation (especially of the gastrointestinal tract or a portion or portions thereof), fever, tissue destruction, shock, and/or death. An inflammatory cytokine response may be characterized by binding of individual cytokines to their cognate cell surface receptor (e.g., IL-34 binding to CSF1R) and subsequent cascades of intracellular signaling that alter cell functions and gene expression. Inflammatory cytokines include, but are not limited to IL-1, IL-6, IL-8, IL-34, and TNF-alpha. Expression of inflammatory cytokines may occur in, for example, macrophages, monocytes, propia lamina mononuclear cells, or other cells of the gastrointestinal tract or cells of the immune system. Methods of inhibiting inflammatory cytokine production include methods that reduce expression levels of some or all inflammatory cytokines in a patient suffering from inflammatory bowel disease. Methods of inhibiting inflammatory cytokine production also include methods that reduce expression levels of some or all inflammatory cytokines in cells of a patient suffering from inflammatory bowel disease.

Methods of the invention for inhibiting inflammatory cytokine production include methods of reducing or inhibiting an IL-34 mediated inflammatory response. An “IL-34 mediated inflammatory response,” as used herein, refers to an inflammatory response initiated, facilitated, or promoted by IL-34 expression or IL-34 signaling activity. An IL-34 mediated inflammatory response may result in expression of inflammatory cytokines including, but not limited to, IL-34, IL-6, IL-8, or TNF-alpha, and activation of inflammatory cytokine signaling. Additionally, an IL-34 mediated inflammatory response may be characterized by granulocyte recruitment, lymphocyte recruitment, systemic inflammation (especially of the gastrointestinal tract or a portion or portions thereof), fever, tissue destruction, shock, and/or death. An IL-34 mediated inflammatory response may also be characterized by activation of IL-34 signaling, for instance, binding of IL-34 to CSF1R and phosphorylation of downstream MAP kinases. Reducing or inhibiting an IL-34 mediated inflammatory response refers to alleviating any or all of the cellular and systemic changes associated with an IL-34 mediated inflammatory response. For example, a reduction in inflammatory cytokine production, immune cell recruitment, or tissue inflammation would indicate reducing or inhibiting of an IL-34 mediated inflammatory response.

The invention also provides methods of inhibiting IL-34 in cells of a patient suffering from an inflammatory bowel disease. IL-34 may be inhibited in any cell in which IL-34 expression or activity occurs, including cells of the gastrointestinal tract, immune system, and blood. Cells of the gastrointestinal tract (including cells of the stomach, duodenum, jejunum, ileum, colon, rectum and anal canal), include columnar epithelial cells, mucosal epithelial cells, zymogenic cells, neck mucus cells, parietal cells, gastrin cells, goblet cells, paneth cells, oligomucus cells, and villus absorptive cells. Cells of the immune system include leukocytes, phagocytes (e.g., macrophages, neutrophils, and dendritic cells), monocytes, mast cells, eosinophils, basophils, natural killer cells, innate cells, lymphocytes, B cells, and T cells. Blood cells include red blood cells (erythrocytes) and white blood cells (leukocytes, monocytes, and platelets).

### IL-34 Inhibition

The invention includes methods of inhibiting IL-34. “Inhibiting IL-34,” as used herein, may refer to a complete or partial reduction in IL-34 expression or activity. Thus, inhibiting IL-34 may refer to alterations in IL-34 gene or chromatin state or altered interaction with regulators of gene transcription or gene accessibility that results in a complete or partial reduction in expression of IL-34 gene products, e.g., IL-34 RNA, IL-34 protein, or peptide sequences of IL-34. Inhibiting IL-34 may also refer to inhibition of processes crucial to IL-34 gene product expression, including, but not limited to IL-34 transcription, IL-34 RNA processing, IL-34 protein translation, or IL-34 post-translational modification. Additionally, inhibiting IL-34 may refer to inhibiting activity of IL-34 gene products, including peptides of IL-34, nucleotide products of IL-34 (e.g., IL-34 mRNA), and IL-34 protein. Inhibiting activity of IL-34 gene products may include a reduction in IL-34 signaling or direct or indirect interaction of IL-34 with other cellular components (e.g., proteins, peptides, DNA, RNA, lipids, or signaling molecules) including nuclear, organelle, cytosolic, membrane, and extracellular components. For example, inhibiting IL-34 activity may include inhibiting IL-34 binding or activation of CSF1R or inhibiting CSF1R downstream signaling effects (e.g., MAP Kinase phosphorylation or macrophage proliferation).

In some embodiments of the invention, an effective amount of an inhibitor of IL-34 may be administered to a patient in need thereof to treat inflammatory bowel disease, to inhibit inflammatory cytokine production in cells of a patient suffering from inflammatory bowel disease, or to reduce or inhibit an IL-34 mediated inflammatory response. An “inhibitor of IL-34,” as used herein, may refer to a reagent capable of inhibiting IL-34 gene expression or gene product activity (i.e., a reagent capable of inhibiting IL-34). Such inhibitors may include, but are not limited to, for example, peptide inhibitors, antibodies, small binding molecules, e.g., natural and synthetic compounds, aptamers, intramers, RNAi (double stranded RNA, siRNA), and anti-IL-34 antisense molecules and oligonucleotides. IL-34 inhibitors may also comprise truncated and/or mutated IL-34 molecules which interfere with IL-34 activity, binding partners, or substrates and which, thereby, inhibit IL-34 function.

Inhibitors of IL-34 include reagents that bind directly to the IL-34 gene or gene products, or components with which IL-34 interacts directly, i.e., components with which the IL-34 gene or gene products has a physical interaction (e.g., CSF1R). Inhibitors of IL-34 would not include reagents that inhibit IL-34 as a consequence of a broad effect on gene expression or gene product activity or as a result of indirect or non-targeted inhibition of IL-34 expression or activity. For instance, an inhibitor of TNF-alpha signaling (e.g., infliximab (Remicade®)) would not be considered an inhibitor of IL-34 because while it indirectly inhibits IL-34 expression, infliximab neither binds IL-34 (the gene or any of its expression products) nor a component with which IL-34 directly and physically interacts.

Inhibitors of IL-34 should have structural and/or functional properties that allow them to exclusively or with a high degree of selectivity act upon a molecular target, for example, IL-34 or an IL-34 binding protein. Thus, an inhibitor of IL-34 should possess the inherent functional property of targeting the IL-34 gene, its RNA or protein products, or another molecular entity whose activity or expression impinges upon the activity or expression of IL-34 or its products either exclusively or with a high degree of specificity. In the case of antibody inhibitors of IL-34, specificity can be engineered into the antibody via inclusion of protein sequences known to bind IL-34 protein epitopes or epitopes of IL-34 signaling partners with a high degree of specificity. In the case of small molecule inhibitors of IL-34, chemical groups can be included in the formulation of the small molecule that allow binding to specific features of IL-34 protein or its signaling partners. Antisense oligonucleotides can be designed such that the targeting portion of the incorporated nucleotide sequence of each antisense oligonucleotide is completely or almost completely complementary to the IL-34 mRNA sequence or the mRNA sequence of an IL-34 interaction partner. Incorporation of such complementary or nearly complementary nucleotide sequences allows one to engineer antisense oligonucleotides with a high degree of specificity for a given target. Peptide inhibitors of IL-34 can include peptide sequences that bind epitopes of IL-34 or its signaling partners or peptide sequences that duplicate or mimic the peptide sequence of IL-34 binding partners, for example peptides that duplicate or mimic the extracellular domain of CSF1R. Small molecule inhibitors of IL-34 can be identified through screening assays to identify small molecules or chemical structures that bind or are likely to bind IL-34 or IL-34 interaction partners. Specificity can be assessed via measurement of parameters such as dissociation constant, or other criteria such as changes in protein or RNA expression levels or other assays that measure IL-34 activity or expression.

“Antisense oligonucleotide,” as used herein, refers to a short synthetic oligonucleotide sequence complementary to the messenger RNA (mRNA), which encodes for the target protein (e.g., IL-34). Antisense oligonucleotide sequences hybridize to the mRNA producing a double-strand hybrid that can lead to the activation of ubiquitary catalytic enzymes, such as RNase H, which degrades DNA/RNA hybrid strands thus preventing protein translation. Antisense oligonucleotides may include small hairpin RNA's (shRNA's), small interfering RNA's (siRNA's), modified antisense oligonucleotides that include 2′-O-alkyl, peptide nucleic acid (PNA), locked nucleic acid (LNA), or morpholino oligomer chemistries. Antisense oligonucleotides may be single stranded or double-stranded oligonucleotides, where only one strand of the oligonucleotide is complementary to the target sequence.

An inhibitor of IL-34 may be a specific inhibitor of IL-34 such as an antisense oligonucleotide or any other means of targeting IL-34 with a high degree of specificity. An antisense oligonucleotide inhibitor of IL-34 may be an oligonucleotide sequence of 5 to 100 oligonucleotides in length, preferably 5 to 90 oligonucleotides in length, preferably 5 to 80 oligonucleotides in length, preferably 5 to 70 oligonucleotides in length, preferably 5 to 60 oligonucleotides in length, preferably 5 to 50 oligonucleotides in length, preferably 5 to 40 oligonucleotides in length, preferably 10 to 30 oligonucleotides in length, or more preferably 8 to 40 oligonucleotides in length. An antisense oligonucleotide inhibitor of IL-34 may be an oligonucleotide sequence complementary to a portion of the IL-34 mRNA sequence (SEQ ID NO: 1).

It is contemplated herein that an antisense oligonucleotide targeting IL-34 may comprise a mixed-backbone wherein the cytosine residues in a CpG pair are replaced by 5′-methylcytosine (abbreviated as Me-dC). Methylphosphonate linkages may also be placed at the 5′ and/or 3′ ends of an antisense oligonucleotide (abbreviated as MeP). The phosphonate backbone of a contemplated IL-34 antisense oligonucleotide may optionally include 1, 2, 3, 4 or more phosphorothioate bonds (e.g., phosphorothioate bonds would replace phosphodiester bonds). In an embodiment, all phosphonate bonds may be phosphorothioate bonds.

It is contemplated that an inhibitor of IL-34 may be an antibody. An antibody inhibitor of IL-34 may be a full-length antibody, a chimeric antibody, a Fab′, a Fab, a F(ab′)2, a single domain antibody (DAB), an Fv, a single chain Fv (scFv), a minibody, a diabody, a triabody, or a mixture thereof In some embodiments, an antibody inhibitor of IL-34 may bind to IL-34, a receptor of IL-34, or another IL-34 interacting protein. In some embodiments, antibody inhibitors of IL-34 may bind to a ligand, prevent binding of the ligand to its binding partner and interrupting the biological response that otherwise would result from the ligand binding to its binding partner. In assessing the binding and specificity of an antigen binding protein, e.g., an antibody or immunologically functional fragment thereof, an antibody or fragment will substantially inhibit binding of a ligand to its binding partner when an excess of antibody reduces the quantity of binding partner bound to the ligand by at least about 20%, 30%, 40%, 50%, 60%, 70%, 80%, 85%, 90%, 95%, 97%, 99% or more (as measured in an in vitro competitive binding assay). Contemplated antibodies may target an epitope, epitopes, or regions of IL-34 or IL-34 interaction partners, for example, CSF1R. In some embodiments, an antibody IL-34 inhibitor will diminish the ability of CSF1R to bind IL-34. Binding of antibodies to CSF1R can, therefore, inhibit IL-34 binding or signaling. Exemplary antibody inhibitors of IL-34 include those described in U.S. Pat. No. 8,182,813, International Patent Application Publication No. WO 2011/123381, International Patent Application Publication No. WO 2011/140249, and International Patent Application Publication No. WO 2011/070024.

It is also contemplated herein that an inhibitor of IL-34 may be a peptide inhibitor. A peptide inhibitor may bind to IL-34, a receptor of IL-34, or another IL-34 interacting protein. In some embodiments, peptide inhibitors of IL-34 may bind to a protein, prevent binding of the protein to its binding partner and interrupting the biological response that otherwise would result from the protein binding to its binding partner. In assessing the binding and specificity of a peptide inhibitor, a peptide inhibitor will substantially inhibit binding of a protein to its binding partner when an excess of peptide inhibitor reduces the quantity of binding partner bound to the protein by at least about 20%, 30%, 40%, 50%, 60%, 70%, 80%, 85%, 90%, 95%, 97%, 99% or more (as measured in an in vitro competitive binding assay). Contemplated peptide inhibitors may target an epitope, epitopes, or regions of IL-34 or IL-34 interaction partners, for example, CSF1R. In some embodiments, a peptide inhibitor IL-34 inhibitor will diminish the ability of CSF1R or other IL-34 binding partners to bind IL-34. Similarly, in some embodiments, a peptide inhibitor IL-34 inhibitor will diminish the ability of IL-34 to bind CSF1R or its other binding partners. In a particular embodiment, a peptide inhibitor of IL-34 includes all or a portion of the extracellular domain of CSF1R, for example, residues 20-517 of NCBI Reference Sequence: NP_005202.2 (SEQ ID NO: 4). Exemplary peptide inhibitors of IL-34 include those described in U.S. Pat. No. 8,183, 207.

### Pharmaceutical Compositions and Routes of Administration

The present invention also provides methods for treating inflammatory bowel disease via administration of a pharmaceutical composition comprising an inhibitor of IL-34. In another aspect, the invention provides a pharmaceutical composition for use in treating inflammatory bowel disease. The pharmaceutical composition may be comprised of an inhibitor of IL-34, such as an antisense oligonucleotide, peptide inhibitor, small molecule, or antibody that targets IL-34 or an IL-34 binding partner, and a pharmaceutically acceptable carrier. As used herein the term “pharmaceutical composition” means, for example, a mixture containing a specified amount of a therapeutic compound, e.g., a therapeutically effective amount, of a therapeutic compound in a pharmaceutically acceptable carrier to be administered to a mammal, e.g., a human, in order to treat inflammatory bowel disease. In some embodiments, contemplated herein are pharmaceutical compositions comprising a contemplated antisense oligonucleotide, peptide inhibitor, small molecule, or antibody inhibitor of IL-34 or an IL-34 binding partner and a pharmaceutically acceptable carrier. In another aspect, the invention discloses use of an inhibitor of IL-34 in the manufacture of a medicament for treating inflammatory bowel disease. “Medicament,” as used herein, has essentially the same meaning as the term “pharmaceutical composition.”

As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art. In one embodiment the pharmaceutical composition is administered orally and includes an enteric coating suitable for regulating the site of absorption of the encapsulated substances within the digestive system or gut. For example, an enteric coating can include an ethylacrylate-methacrylic acid copolymer.

In one embodiment, a contemplated inhibitor of IL-34 and any pharmaceutical composition thereof may be administered by one or several routes, including topically, parenterally, orally, pulmonarily, intratracheally, intranasally, transdermally, or intraduodenally. The term parenteral as used herein includes subcutaneous injections, intrapancreatic administration, intravenous, intramuscular, intraperitoneal, intrasternal injection or infusion techniques. For example, an inhibitor of IL-34 may be administered subcutaneously to a subject. In another example, an IL-34 inhibitor may be administered orally to a subject. In another example, the inhibitor of IL-34 may be administered directly to the gastrointestinal system, or specific regions of the gastrointestinal system (e.g., the ileum, colon, or rectum) via parenteral administration.

Pharmaceutical compositions containing an inhibitor of IL-34, such as those disclosed herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

Parenteral Administration

The pharmaceutical compositions of the invention can be formulated for parenteral administration, e.g., formulated for injection via the intravenous, intramuscular, subcutaneous, intralesional, or intraperitoneal routes. The preparation of an aqueous composition, such as an aqueous pharmaceutical composition containing an IL-34 inhibitor, will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

Solutions of active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. In addition, sterile, fixed oils may be employed as a solvent or suspending medium. For this purpose any bland fixed oil can be employed including synthetic mono- or diglycerides. In addition, fatty acids such as oleic acid can be used in the preparation of injectables. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In one embodiment, the inhibitor of IL-34 may be suspended in a carrier fluid comprising 1% (w/v) sodium carboxymethylcellulose and 0.1% (v/v) TWEEN™80. Under ordinary conditions of storage and use, these preparations contain a preservative to prevent the growth of microorganisms.

Injectable preparations, for example, sterile injectable aqueous or oleaginous suspensions may be formulated according to the known art using suitable dispersing or wetting agents and suspending agents. Generally, dispersions are prepared by incorporating the various sterilized active ingredients into a sterile vehicle which contains the basic dispersion medium and the required other ingredients from those enumerated above. Sterile injectable solutions of the invention may be prepared by incorporating an inhibitor of IL-34 in the required amount of the appropriate solvent with various of the other ingredients enumerated above, as required, followed by filtered sterilization. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The injectable formulations can be sterilized, for example, by filtration through a bacteria-retaining filter.

The preparation of more, or highly concentrated solutions for intramuscular injection is also contemplated. In this regard, the use of DMSO as solvent is preferred as this will result in extremely rapid penetration, delivering high concentrations of the inhibitor of IL-34 to a small area.

Suitable preservatives for use in such a solution include benzalkonium chloride, benzethonium chloride, chlorobutanol, thimerosal and the like. Suitable buffers include boric acid, sodium and potassium bicarbonate, sodium and potassium borates, sodium and potassium 10 carbonate, sodium acetate, sodium biphosphate and the like, in amounts sufficient to maintain the pH at between about pH 6 and pH 8, and preferably, between about pH 7 and pH 7.5. Suitable tonicity agents are dextran 40, dextran 70, dextrose, glycerin, potassium chloride, propylene glycol, sodium chloride, and the like, such that the sodium chloride equivalent of the solution is in the range 0.9 plus or minus 0.2%. Suitable antioxidants and stabilizers include sodium bisulfate, sodium metabisulfite, sodium thiosulfite, thiourea and the like. Suitable wetting and clarifying agents include polysorbate 80, polysorbate 20, poloxamer 282 and tyloxapol. Suitable viscosity-increasing agents include dextran 40, dextran 70, gelatin, glycerin, hydroxyethylcellulose, hydroxymethylpropylcellulose, lanolin, methylcellulose , petrolatum, polyethylene glycol, polyvinyl alcohol, polyvinylpyrrolidone, carboxymethylcellulose and the like.

Oral Administration

In some embodiments, contemplated herein are compositions suitable for oral delivery of an IL-34 inhibitor, e.g., tablets that include an enteric coating, e.g., a gastro-resistant coating, such that the compositions may deliver the IL-34 inhibitor to, e.g., the gastrointestinal tract of a patient. For example, such administration may result in a topical effect, substantially topically applying the IL-34 inhibitor directly to an affected portion of the gastrointestinal tract of a patient. Such administration, may, in some embodiments, substantially avoid unwanted systemic absorption of the IL-34 inhibitor.

For example, a tablet for oral administration is provided that comprises granules (e.g., is at least partially formed from granules) that include a disclosed IL-34 inhibitor, e.g., an IL-34 antisense oligonucleotide, and pharmaceutically acceptable excipients. Such a tablet may be coated with an enteric coating. Contemplated tablets may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated pharmaceutical formulations include an intra-granular phase that includes a contemplated IL-34 inhibitor, e.g. an IL-34 or IL-34 interaction partner antisense oligonucleotide, peptide inhibitor, small molecule, or antibody, or a pharmaceutically acceptable salt, e.g. an IL-34 or IL-34 interaction partner antisense oligonucleotide, peptide inhibitor, small molecule, or antibody, and a pharmaceutically acceptable filler. For example, an IL-34 or IL-34 interaction partner antisense oligonucleotide, peptide inhibitor, small molecule, or antibody and a filler may be blended together, optionally, with other excipients, and formed into granules. In some embodiments, the intragranular phase may be formed using wet granulation, e.g. a liquid (e.g., water) is added to the blended IL-34 inhibitor compound and filler, and then the combination is dried, milled and/or sieved to produce granules. One of skill in the art would understand that other processes may be used to achieve an intragranular phase.

In some embodiments, contemplated formulations include an extra-granular phase, which may include one or more pharmaceutically acceptable excipients, and which may be blended with the intragranular phase to form a disclosed formulation.

A disclosed formulation may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pregelatinized starch, calcium carbonate, and others including combinations thereof.

In some embodiments, a disclosed formulation may include a intragranular phase and/or a extragranular phase that includes a binder, which may generally function to hold the ingredients of the pharmaceutical formulation together. Exemplary binders of the invention may include, but are not limited to, the following: starches, sugars, cellulose or modified cellulose such as hydroxypropyl cellulose, lactose, pregelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated formulations, e.g., that include an intragranular phase and/or an extragranular phase, may include a disintegrant such as but are not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extragranular phase may include a disintegrant.

In some embodiments, a contemplated formulation includes an intra-granular phase comprising an inhibitor of IL-34 such as an antisense oligonucleotide, small molecule inhibitor, peptide inhibitor, or antibody and excipients chosen from: mannitol, microcrystalline cellulose, hydroxypropylmethyl cellulose, and sodium starch glycolate or combinations thereof, and an extra-granular phase comprising one or more of: microcrystalline cellulose, sodium starch glycolate, and magnesium stearate or mixtures thereof.

In some embodiments, a contemplated formulation may include a lubricant, e.g. an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In some embodiments, the pharmaceutical formulation comprises an enteric coating. Generally, enteric coatings create a barrier for the oral medication that controls the location at which the drug is absorbed along the digestive track. Enteric coatings may include a polymer that disintegrates at different rates according to pH. Enteric coatings may include for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

Exemplary enteric coatings include Opadry® AMB, Acryl-EZE , Eudragit® grades. In some embodiments, an enteric coating may comprise about 5% to about 10%, about 5% to about 20%, 8 to about 15%, about 8% to about 20%, about 10% to about 20%, or about 12 to about 20%, or about 18% of a contemplated tablet by weight. For example, enteric coatings may include an ethylacrylate-methacrylic acid copolymer.

For example, in a contemplated embodiment, a tablet is provided that comprises or consists essentially of about 0.5% to about 70%, e.g. about 0.5% to about 10%, or about 1% to about 20%, by weight of an IL-34 inhibitor or a pharmaceutically acceptable salt thereof (e.g. an IL-34 or IL-34 interaction partner antisense oligonucleotide, antibody, small molecule, or peptide inhibitor). Such a tablet may include for example, about 0.5% to about 60% by weight of mannitol, e.g. about 30% to about 50% by weight mannitol, e.g. about 40% by weight mannitol; and/or about 20% to about 40% by weight of microcrystalline cellulose, or about 10% to about 30% by weight of microcrystalline cellulose. For example, a disclosed tablet may comprise an intragranular phase that includes about 30% to about 60%, e.g. about 45% to about 65% by weight, or alternatively, about 5 to about 10% by weight of an IL-34 inhibitor, about 30% to about 50%, or alternatively, about 5% to about 15% by weight mannitol, about 5% to about 15% microcrystalline cellulose, about 0% to about 4%, or about 1% to about 7% hydroxypropylmethylcellulose, and about 0% to about 4%, e.g. about 2% to about 4% sodium starch glycolate by weight.

In another contemplated embodiment, a pharmaceutical tablet formulation for oral administration of an IL-34 inhibitor comprises an intra-granular phase, wherein the intra-granular phase includes an IL-34 inhibitor such as an IL-34 or IL-34 interaction partner antisense oligonucleotide, antibody, small molecule, or peptide inhibitor, or a pharmaceutically acceptable salt thereof (such as a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof The pharmaceutical composition may also include an enteric coating of about 12% to 20% by weight of the tablet. For example, a pharmaceutically acceptable tablet for oral use may comprise about .5% to 10% by weight of an IL-34 inhibitor, e.g., an IL-34 or IL-34 interaction partner antisense oligonucleotide, antibody, small molecule, or peptide inhibitor, or a pharmaceutically acceptable salt thereof, about 30% to 50% by weight mannitol, about 10% to 30% by weight microcrystalline cellulose, and an enteric coating comprising an ethylacrylate-methacrylic acid copolymer.

In another example, a pharmaceutically acceptable tablet for oral use may comprise an intra-granular phase, comprising about 5 to about 10% by weight of an IL-34 inhibitor, e.g., an IL-34 or IL-34 interaction partner antisense oligonucleotide, antibody, small molecule, or peptide inhibitor, or a pharmaceutically acceptable salt thereof, about 40% by weight mannitol, about 8% by weight microcrystalline cellulose, about 5% by weight hydropropylmethyl cellulose, and about 2% by weight sodium starch glycolate; an extra-granular phase comprising about 17% by weight microcrystalline cellulose, about 2% by weight sodium starch glycolate, about 0.4% by weight magnesium stearate; and an enteric coating over the tablet comprising an ethylacrylate-methacrylic acid copolymer.

In some embodiments the pharmaceutical composition may contain an enteric coating comprising about 13% or about 15%, 16%, 17% or 18% by weight, e.g., AcyrlEZE® (see, e.g., PCT Publication No. WO2010/054826, which is hereby incorporated by reference in its entirety).

The rate at which point the coating dissolves and the active ingredient is released is its dissolution rate. In an embodiment, a contemplated tablet may have a dissolution profile, e.g. when tested in a USP/EP Type 2 apparatus (paddle) at 100 rpm and 37° C. in a phosphate buffer with a pH of 7.2, of about 50% to about 100% of the inhibitor of IL-34 releasing after about 120 minutes to about 240 minutes, for example after 180 minutes. In another embodiment, a contemplated tablet may have a dissolution profile, e.g. when tested in a USP/EP Type 2 apparatus (paddle) at 100 rpm and 37° C. in diluted HCl with a pH of 1.0, where substantially none of the inhibitor of IL-34 is released after 120 minutes. A contemplated tablet, in another embodiment, may have a dissolution profile, e.g. when tested in USP/EP Type 2 apparatus (paddle) at 100 rpm and 37° C. in a phosphate buffer with a pH of 6.6, of about 10% to about 30%, or not more than about 50%, of the inhibitor of IL-34 releasing after 30 minutes.

Contemplated formulations, e.g. tablets, in some embodiments, when orally administered to the patient may result in minimal plasma concentration of the IL-34 inhibitor in the patient. In another embodiment, disclosed formulations, when orally administered to a patient, topically deliver to the colon or rectum of a patient, e.g. to an affected or diseased site of a patient.

In some embodiments, methods provided herein may further include administering at least one other agent that is directed to treatment of diseases and disorders disclosed herein. In one embodiment, contemplated other agents may be co-administered (e.g., sequentially or simultaneously).

Agents contemplated include immunosuppressive agents including glucocorticoids, cytostatics, antibodies, agents acting on immunophilins, interferons, opioids, TNF binding proteins, mycophenolate, and small biological agents. For example, contemplated immunosuppressive agents include, but are not limited to: tacrolimus, cyclosporine, pimecrolimus, sirolimus, everolimus, mycophenolic acid, fingolimod, dexamethasone, fludarabine, cyclophosphamide, methotrexate, azathioprine, leflunomide, teriflunomide, anakinra, anti-thymocyte globulin, anti-lymphocyte globulin, muromonab-CD3, afutuzumab, rituximab, teplizumab, efalizumab, daclizumab, basiliximab, adalimumab, infliximab, certolizumab pegol, natalizumab, and etanercept. Other contemplated agents include antibiotics, anti-diarrheals, laxatives, pain relievers, iron supplements, and calcium or vitamin D or B-12 supplements.

### Dosage and Frequency of Administration

Exemplary formulations include dosage forms that include or consist essentially of about 35 mg to about 500 mg of an IL-34 inhibitor. For example, formulations that include about 35 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, or 250 mg of an IL-34 inhibitor are contemplated herein. In one embodiment, a formulation may include about 40 mg, 80 mg, or 160 mg of an IL-34 inhibitor. In some embodiments, a formulation may include at least 100 μg of an IL-34 inhibitor. For example, formulations may include about 0.1 mg, 0.2 mg, 0.3 mg, 0.4 mg, 0.5 mg, 1 mg, 5 mg, 10 mg, 15 mg, 20 mg, or 25 mg of an IL-34 inhibitor. The amount administered will depend on variables such as the type and extent of disease or indication to be treated, the overall health and size of the patient, the in vivo potency of the IL-34 inhibitor, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study designed to run from 40 mg to 160 mg. Dosing frequency can vary, depending on factors such as route of administration, dosage amount and the disease being treated. Exemplary dosing frequencies are once per day, once per week and once every two weeks. In some embodiments, dosing is once per day for 7 days.

### Diagnostic Methods of the Invention

The invention also provides a method of diagnosing a patient with inflammatory bowel disease that relies upon detecting levels of IL-34 expression signal in one or more biological samples of a patient. As used herein, the term “IL-34 expression signal” can refer to any indication of IL-34 gene expression, or gene or gene product activity. IL-34 gene products include RNA (e.g., mRNA), peptides, and proteins. Indices of IL-34 gene expression that can be assessed include, but are not limited to, IL-34 gene or chromatin state, IL-34 gene interaction with cellular components that regulate gene expression, IL-34 gene product expression levels (e.g., IL-34 RNA expression levels, IL-34 protein expression levels), or interaction of IL-34 RNA or protein with transcriptional, translational, or post-translational processing machinery. Indices of IL-34 gene product activity include, but are not limited to, assessment of IL-34 signaling activity (e.g., assessment of CSF1R activation or MAPK1/MAPK3 phosphorylation) and assessment of IL-34 receptor binding (e.g., CSF1R binding).

Detection of IL-34 expression signal may be accomplished through in vivo, in vitro, or ex vivo methods. In a preferred embodiment, methods of the invention may be carried out in vitro. Methods of detecting may involve detection in blood, serum, fecal matter, tissue, or cells of a patient. Detection may be achieved by measuring IL-34 expression signal in whole tissue, tissue explants, cell cultures, dissociated cells, cell extract, or body fluids, including blood or serum. Contemplated methods of detection include assays that measure levels of IL-34 gene product expression such as Western blotting, FACS, ELISA, other quantitative binding assays, cell or tissue growth assays, Northern blots, quantitative or semi-quantitative polymerase chain reaction, medical imaging methods (e.g., MRI), or immunostaining methods (e.g., immunohistochemistry or immunocytochemistry).

### EXAMPLES

The invention is further illustrated by the following examples. The examples are provided for illustrative purposes only, and are not to be construed as limiting the scope or content of the invention in any way.

### Example 1

### IL-34 Expression in Inflammatory Bowel Disease

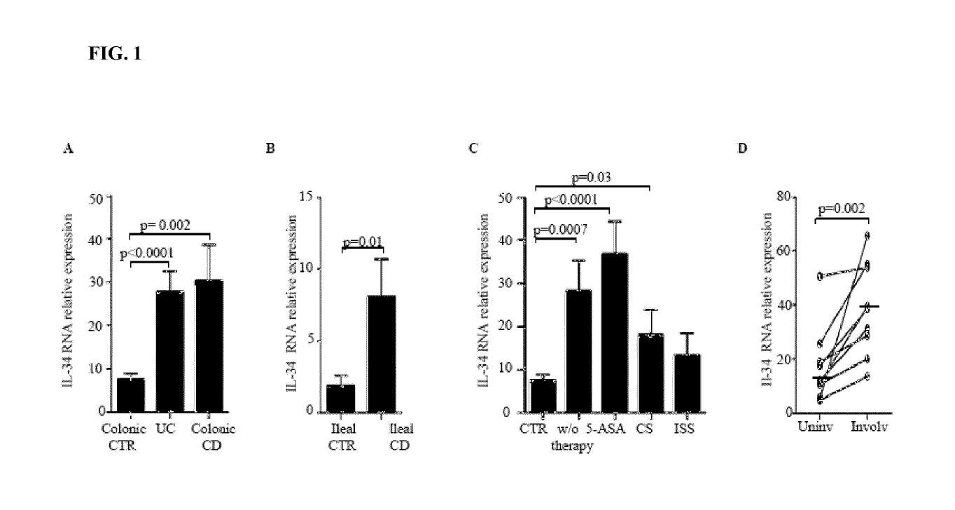

Expression of IL-34 mRNA transcripts was analyzed in colonic biopsies taken from patients suffering from inflammatory bowel diseases and control patients. Colonic biopsies were collected from 14 control patients (Colonic CTR), 22 patients with ulcerative colitis (UC), and 15 patients with Crohn's disease (Colonic CD; FIG. 1A). RNA was collected and real-time PCR was performed to detect levels of IL-34 mRNA. Levels of IL-34 expression signal were normalized to β-actin levels. Analysis of IL-34 expression signal was significantly elevated in tissue harvested from Crohn's disease and ulcerative colitis patients compared to control samples (FIG. 1A; Colonic CTR v. UC, p<0.0001; Colonic CTR v. Colonic CD, p=0.002; mean±SEM). Similarly, IL-34 mRNA transcript expression was evaluated in ileal biopsies taken from three control patients (Ileal CTR) and seven patients suffering from Crohn's disease (Ileal CD; FIG. 1B). RNA was collected from biopsies, real-time PCR was performed, and levels of IL-34 expression signal were normalized to β-actin levels. Analysis of IL-34 expression signal was significantly elevated in ileal tissue harvested from Crohn's disease patients compared to control samples (FIG. 1B; p<0.0001; mean±SEM). These results demonstrate that levels of IL-34 mRNA transcript expression were increased in tissue from patients suffering from inflammatory bowel disease as compared to control patients.

IL-34 mRNA transcript expression was also analyzed in colonic biopsies from patients suffering from inflammatory bowel disease undergoing different therapeutic treatments (FIG. 1C). Colonic biopsies were harvested from 14 control patients (CTR), 9 inflammatory bowel disease patients not receiving therapy (6 ulcerative colitis patients and 3 Crohn's disease patients; w/o therapy), 17 inflammatory bowel disease patients treated with mesalamine (11 ulcerative colitis patients and 6 Crohn's disease patients; 5-ASA), 6 inflammatory bowel disease patients treated with steroids (5 ulcerative colitis patients and 1 Crohn's disease patient; CS), and 5 inflammatory bowel disease patients treated with immunomodulators (1 ulcerative colitis patient and 4 Crohn's disease patients; ISS). Tissue biopsies were then analyzed for IL-34 mRNA expression by real-time PCR. Levels of IL-34 mRNA expression signal were normalized to β-actin mRNA expression signal. Comparison of IL-34 mRNA expression across samples demonstrated that a significant difference existed between control samples, patients not treated for inflammatory bowel disease, patients treated with mesalamine, and patients treated with steroids (FIG. 1C; CTR v. w/o therapy, p=0.0007; CTR v. 5-ASA, p<0.0001; CTR v. CS, p=0.03; mean±SEM). These findings demonstrate that high levels of IL-34 expression persisted in many patients suffering from inflammatory bowel disease even after treatment with available therapies.

In addition, IL-34 RNA expression was compared between paired biopsies taken from individual patients. Biopsies were harvested from uninvolved (Uninv) and involved (Involv) mucosa of 9 patients suffering from inflammatory bowel disease (7 ulcerative colitis patients and 2 Crohn's disease patients). Real-time PCR was used to evaluate IL-34 mRNA expression in biopsied tissue, and levels of IL-34 signal were normalized to β-actin signal. IL-34 mRNA expression was found to be significantly lower in uninvolved tissue compared to involved tissue (FIG. 1D; p=0.002; horizontal bars indicate median values for each group). This result demonstrates that IL-34 mRNA expression was significantly lower in areas of the gastrointestinal tract not affected by inflammatory bowel disease compared to areas of the gastrointestinal tract affected by inflammatory bowel disease in the same patient.

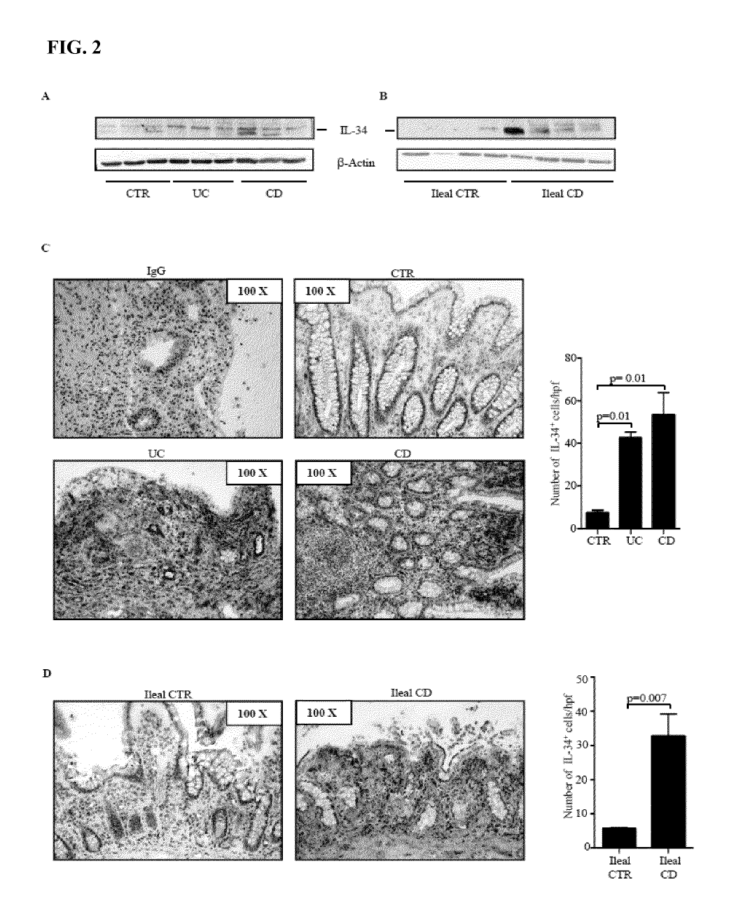

Expression of IL-34 protein was analyzed in biological samples harvested from colonic mucosa of patients suffering from inflammatory bowel disease. Total protein extract was collected from colonic mucosal samples harvested from 3 control patients (CTR), 3 ulcerative colitis patients (UC), and 3 Crohn's disease patients (CD), and samples were analyzed by Western blot for IL-34 protein signal (FIG. 2A). β-actin was used as a control. Extracts from inflammatory bowel disease patients displayed elevated levels of IL-34 signal compared to controls. Similarly, total protein extract from ileal mucosal samples harvested from 4 control (Ileal CTR) and 4 Crohn's disease (Ileal CD) patients were analyzed by Western blot for IL-34 protein signal (FIG. 2B). β-actin was again used as a control. IL-34 signal as detected in Ileal CD protein extract was elevated compared to control samples. These results demonstrated that an overall elevated level of IL-34 protein exists in cells of patients suffering from inflammatory bowel disease.

Immunohistochemistry was also used to analyze expression of IL-34 protein in patients suffering from inflammatory bowel disease. Colonic tissue samples were stained using either isotype control antibody (IgG, upper left panel) or IL-34 antibody (FIG. 2C). Photomicrographs of sections stained with IL-34 antibody showed a clear increase in IL-34 signal in tissue sections harvested from patients with ulcerative colitis (UC) and Crohn's disease (CD) compared to control samples (CTR). Furthermore, analysis of the number of IL-34-positive cells per high power field (IL-34⁺ cells/hpf) showed that there was a significant increase in the number of IL-34⁺ cells/hpf in UC and CD samples compared to controls (FIG. 2C, right panel; 6 CTR v. 3 UC patients, p=0.01; 6 CTR v. 3 CD patients, p=0.01; mean±SEM). Similarly, ileal tissue samples from control patients (Ileal CTR) and Crohn's disease patients (Ileal CD) were stained with IL-34 antibody (FIG. 2D). Photomicrographs of ileal sections stained with IL-34 antibody showed a clear increase in IL-34 signal in Ileal CD tissue sections compared to Ileal CTR samples. Analysis of ileal tissue sections showed that there was also a significant increase in the number of IL-34⁺cells/hpf in Ileal CD samples compared to controls (FIG. 2D, right panel; 4 CTR v. 5 CD patients, p=0.007; mean±SEM). These results demonstrated a clear elevation in overall tissue expression levels of IL-34 protein in patients with inflammatory bowel disease as well as a significant increase in the number of individual cells that expressed IL-34 in such patients.

### Example 2

### CSF1R Expression is Elevated in Inflammatory Bowel Disease and Colon Cancer Cell Lines

Expression of the IL-34 receptor CSF1R was examined in colonic tissue from IBD and control patients. Biopsy samples were taken from inflamed mucosa of 8 patients with colonic CD and 14 patients with UC undergoing colonoscopy for a clinically active disease. Within this group, biopsies were collected at the time of initial diagnosis from 3 patients with UC who were not receiving drugs; 12 patients (4 patients with colonic CD and 8 patients with UC) receiving mesalamine; 4 patients (2 patients with colonic CD and 2 patients with UC) receiving steroids; and 3 patients (2 patients with colonic CD and 1 patient with UC) receiving immunosuppressive drugs. Biopsies were also collected from 5 patients with colonic CD and 10 patients with UC undergoing surgery for a chronic active disease poorly responsive to medical treatment. Control colonic biopsy samples included samples taken from 9 subjects with irritable bowel syndrome and mucosal specimens taken from macroscopically and microscopically unaffected colonic areas of 5 patients undergoing surgery for colon cancer.

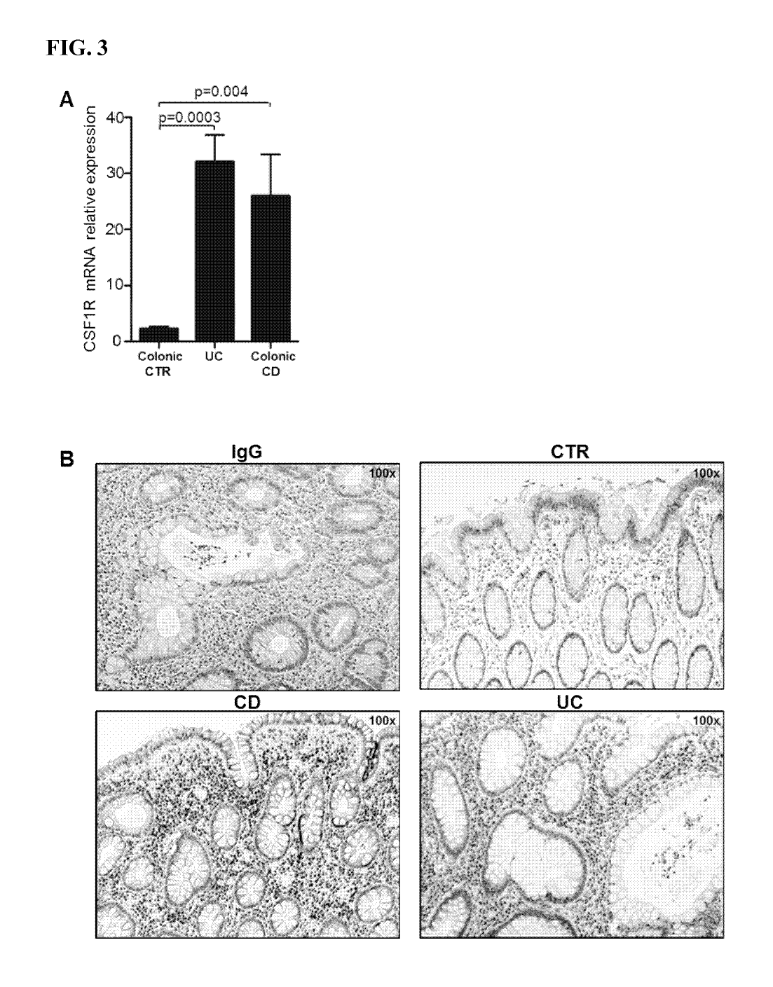

Total RNA extracts were collected from the colonic biopsies of IBD patients and controls described above and CSF1R mRNA transcript expression was analyzed by real-time PCR. Levels of CSF1R signal were normalized to β-actin signal. CSF1R RNA transcripts were increased in inflamed colon of both UC and CD (colonic CD) patients relative to controls (Colonic CTR) (FIG. 3A; Colonic CTR vs. UC, p−0.0003; Colonic CTR vs. Colonic CD, p=0.004). CSF1R RNA expression did not differ between IBD patients taking no drug or mesalazine and IBD patients on steroids or immunosuppressive drugs (data not shown). These results demonstrate that CSF1R mRNA transcript expression was significantly higher in colonic tissue of UC and CD patients relative to colonic tissue from control patients not suffering from IBD.

Expression of CSF1R protein levels in colonic tissue was also analyzed by immunohistochemistry. Immunohistochemical staining of colonic tissue sections was performed using a rabbit monoclonal antibody directed against human CSF1R (Novus Biological). Tissue sections were counterstained with haematoxylin. Isotype control IgG-stained sections were prepared using a purified mouse normal IgG control antibody (R&D Systems; FIG. 3B, IgG)) in place of the CSF1R antibody. Epithelial cells of colonic tissue sections from control samples (CTR) expressed CSF1R, while little CSFR staining was observed in the regions of the crypts and lamina propria of control samples (FIG. 3B). By contrast, CSF1R staining was observably more widespread in both the epithelial and lamina propria compartments in tissue sections from IBD patients, and intense CSF1R staining was observed in the crypts of IBD tissue sections (FIG. 3B, compare IgG to CD and UC panels). These results demonstrate that CSF1R expression was observably more widespread and intense in colonic tissue of IBD patients compared to patients not suffering from IBD.

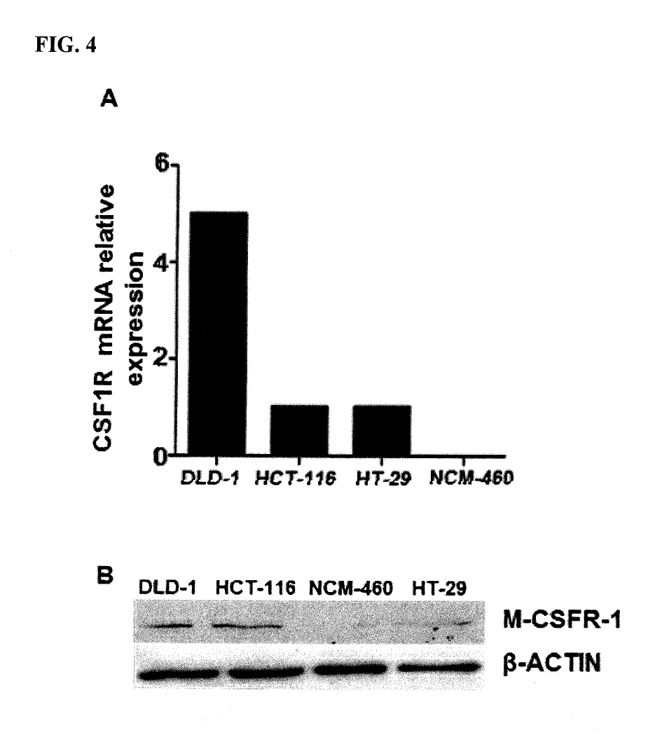

CSF1R expression was also analyzed in a series of colon cancer epithelial cell lines. Colon cancer epithelial cell lines (DLD-1, HT-29, and HCT-116) and a control colon epithelial cell line (NCM460) were cultured in plastic flasks and CSF1R RNA and protein were evaluated in these cells by real-time PCR and Western blotting, respectively. Constitutive CSF1R mRNA transcript expression was observed in the cancer cell lines, while constitutive expression was not observed in control cells (FIG. 4A). Similarly, Western blotting revealed constitutive CSF1R protein expression in all of the colon cancer epithelial cell lines, while CSF1R protein expression was barely detectable in the normal colonic epithelial cell line NCM460 (FIG. 4B). These results demonstrate that CSF mRNA and protein expression was observably higher in colon cancer epithelial cells relative to non-cancerous control cells.

### Example 3

### TNF-alpha Regulates IL-34 Expression

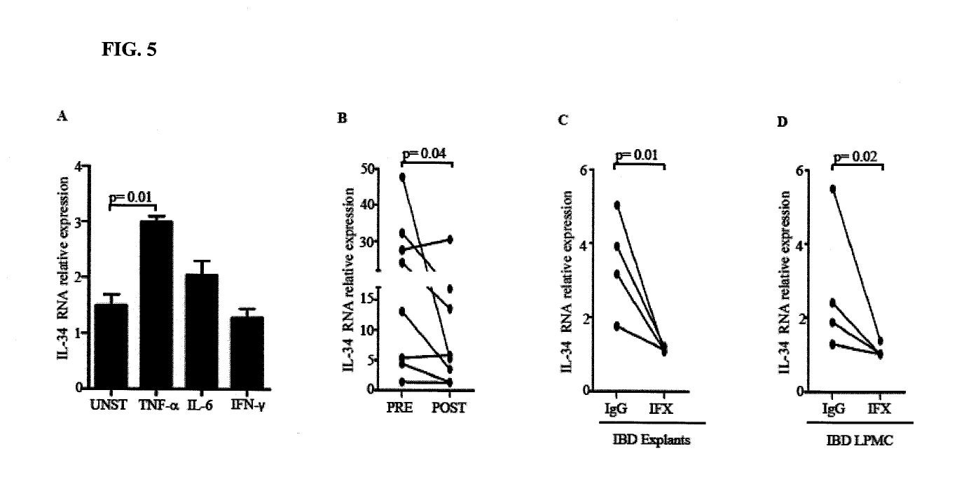

The effect of various factors on IL-34 expression was analyzed by culturing normal colonic mucosal explants without stimulation (UNST) or in the presence of TNF-alpha (TNF-α, 20 ng/ml), IL-6 (50 ng/ml), or interferon gamma (IFN-γ, 100 ng/ml). Explants were exposed to these factors for 6 hours. IL-34 mRNA expression was then analyzed by real-time PCR. Levels of IL-34 signal were normalized to β-actin signal. Comparison of UNST samples to explants exposed to TNF-α showed that there was a significant increase in IL-34 mRNA expression in the presence of TNF-α (FIG. 5A; UNST v. TNF-α, p=0.01; mean±SEM of 2 experiments). These results indicate that TNF-α was able to stimulate IL-34 expression in colonic mucosal explants.

To investigate the effect of TNF-alpha inhibition on IL-34 expression, colonic mucosal biopsies were taken from individual IBD patients before (PRE) and after (POST) treatment with a monoclonal antibody directed against TNF-alpha known as infliximab (Remicade®). IL-34 RNA expression in colonic tissue was measured by real-time PCR, and β-actin RNA signal was used for normalization. Pairs of PRE and POST data points in FIG. 3B connected by individual lines represent data taken from individual patients. Analysis of IL-34 mRNA expression signal after treatment with infliximab (Remicade®) demonstrated a significant decrease compared to pre-treatment levels (FIG. 5B; p=0.04; mean±SEM of all experiments). These results demonstrate that inhibition of TNF-alpha activity was sufficient to reduce levels of IL-34 expression in colonic tissue from patients with inflammatory bowel disease.

To further investigate the effect of TNF-alpha inhibition on IL-34 expression, colonic mucosal biopsies (IBD Explants) taken from 4 inflammatory bowel disease patients (1 ulcerative colitis patient and 3 Crohn's disease patients) were cultured in the presence of infliximab (Remicade®, (IFX)), or control IgG (IgG) for 24 hours. IL-34 RNA expression was then measured by real-time PCR, using β-actin signal for normalization. Analysis of IL-34 mRNA expression signal demonstrated a significant decrease in IBD explants cultured in the presence of IFX as opposed to IgG (FIG. 5C; p=0.01; mean±SEM of all experiments). Additionally, colonic lamina propria mononuclear cells (IBD LPMC) isolated from surgical specimens of 4 inflammatory bowel disease patients (1 ulcerative colitis patient and 3 Crohn's disease patients) were cultured were cultured in the presence of IFX or IgG for 18 hours, after which point IL-34 RNA expression was measured by real-time PCR. β-actin signal was again used for normalizing IL-34 RNA signal. Analysis of IL-34 mRNA expression signal showed a significant decrease in IBD LPMC cultured in the presence of IFX as opposed to IgG (FIG. 5D; p=0.02; mean±SEM of all experiments). These results clearly demonstrate that inhibition of TNF-alpha activity was sufficient to reduce levels of IL-34 expression in cells and tissue from patients with inflammatory bowel disease.

### Example 4

### IL-34 Stimulates Inflammatory Cytokine Production

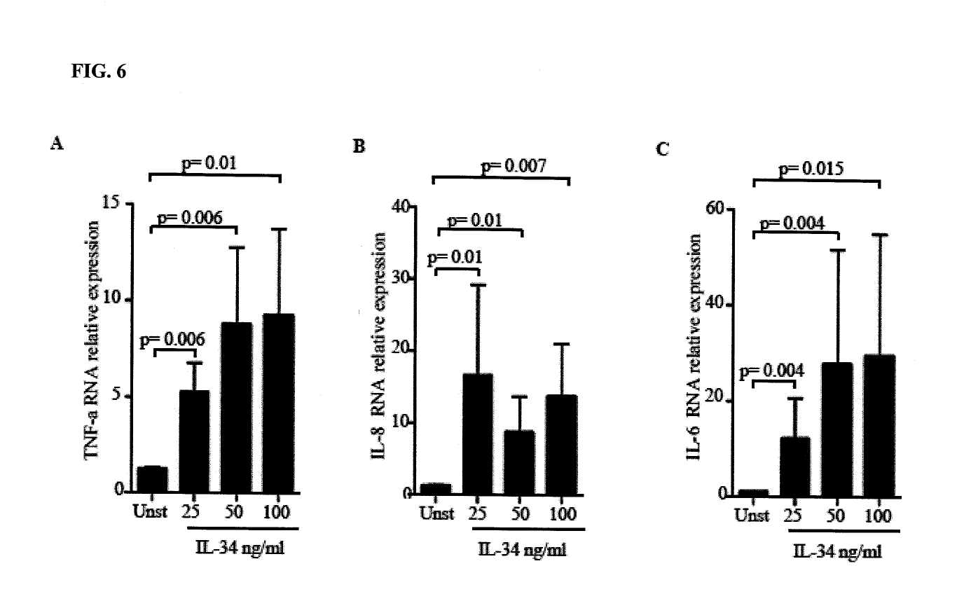

The effect of IL-34 stimulation on inflammatory cytokine production was investigated. Normal colonic lamina propria mononuclear cells were cultured in the absence of recombinant IL-34 (Unst) or in the presence of increasing amounts of recombinant human IL-34 (25, 50, or 100 ng/ml recombinant IL-34). Cultures were exposed to recombinant IL-34 for 6 hours, after which relative mRNA expression levels of the inflammatory cytokines TNF-alpha (FIG. 6A), IL-8 (FIG. 6B), and IL-6 (FIG. 6C) were analyzed. Detection of mRNA signal was performed using real-time PCR, and all signals were normalized to β-actin. In all instances, a significant increase in inflammatory cytokine production was observed compared to Unst samples, in the presence of IL-34 stimulation. Data shown in each panel of FIG. 4 indicates the mean±SEM of 5 independent experiments. These findings demonstrate that IL-34 stimulation was sufficient to stimulate inflammatory cytokine production, and suggests that IL-34 may play an important role in stimulating inflammatory cytokine production and an inflammatory response in inflammatory bowel disease.

### Example 5

### IL-34 Stimulates CCL20 Expression Via an ERK-Dependent Pathway

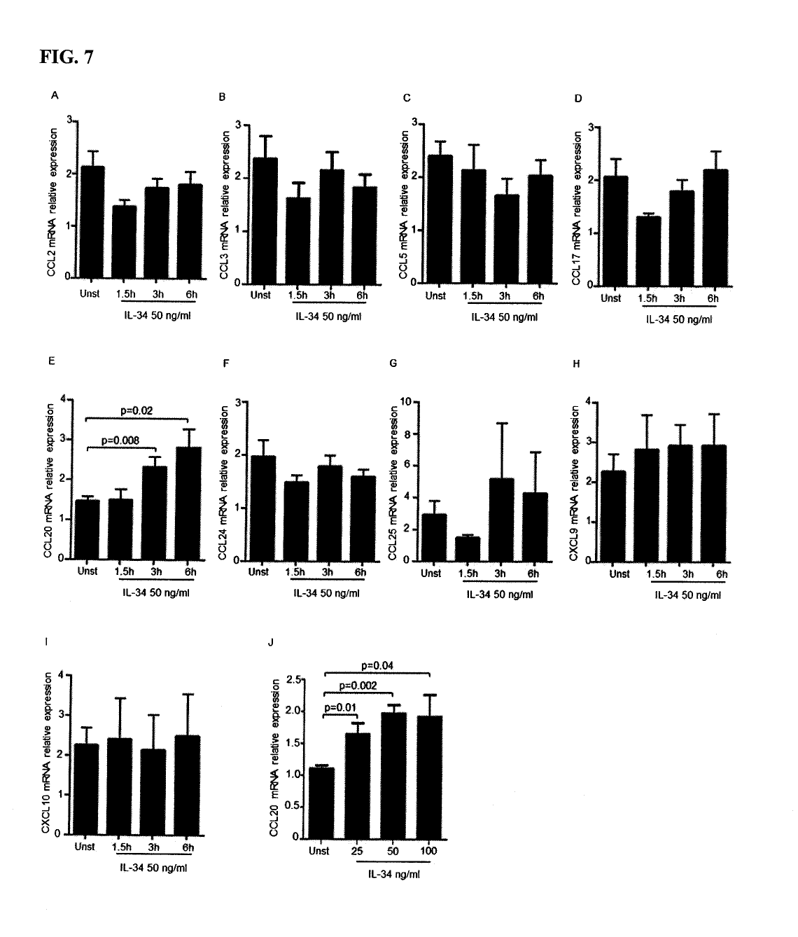

The ability of IL-34 to stimulate expression of various chemokines in the colon cancer epithelial cell line DLD-1 was analyzed. Briefly, 2×10⁵DLD-1 cells were plated into 6-well plates, left to adhere for 24 hours, and starved for 12 hours. Cells were either left unstimulated (Unst) or stimulated with recombinant human IL-34 (R&D Systems, Inc. Minneapolis, Minn.) at 50 ng/ml for 1.5, 3, or 6 hours. Expression of mRNA transcripts of various chemokines—including Chemokine Ligand (CCL)2, CCL3, CCLS, CCL17, CCL20, CCL24, CCL25, C-X-C motif chemokine (CXCL)9, and CXCL10 —believed to contribute to IBD-related inflammation was evaluated by real-time PCR. IL-34 exposure stimulated increased expression of CCL20 mRNA, but failed to stimulate increased expression of other chemokines in DLD-1 cells (FIG. 7, panels A-I). Furthermore, CCL20 mRNA expression was increased in DLD-1 cells following stimulation with IL-34 at all concentrations tested (25 ng/ml, p=0.01; 50 ng/ml, p=0.002; and 100 ng/ml, p=0.04; FIG. 7, panel J). In all instances, levels of chemokine expression were normalized to β-actin signal. These results demonstrate that IL-34 selectively stimulated increased CCL20 chemokine expression in epithelial cells.

To investigate the ability of IL-34 to regulate CCL20 expression in colonic tissue, mucosal explants taken from 3 IBD patients were each cultured with a neutralizing IL-34 antibody (Anti-IL-34) or a control antibody (IgG) for 20 hours. Culture supernatants were collected and CCL20 expression was measured by ELISA. Treatment with anti-IL-34 antibody reduced CCL20 secretion (Table 1). These results demonstrate that blocking IL-34 activity in colonic mucosal cells of IBD patients suppressed CCL20 expression and release.

TABLE 1
IL-34 neutralization decreases CCL20 production in IBD mucosal explants

| | IgG | Anti-IL-34 |
|---|---|---|
| Experiment | (pg/ml CCL20) | (pg/ml CCL20) |
| 1 | 584.93 | 308.1 |
| 2 | 44.36 | 24.70 |
| 3 | 12.97 | 5.34 |

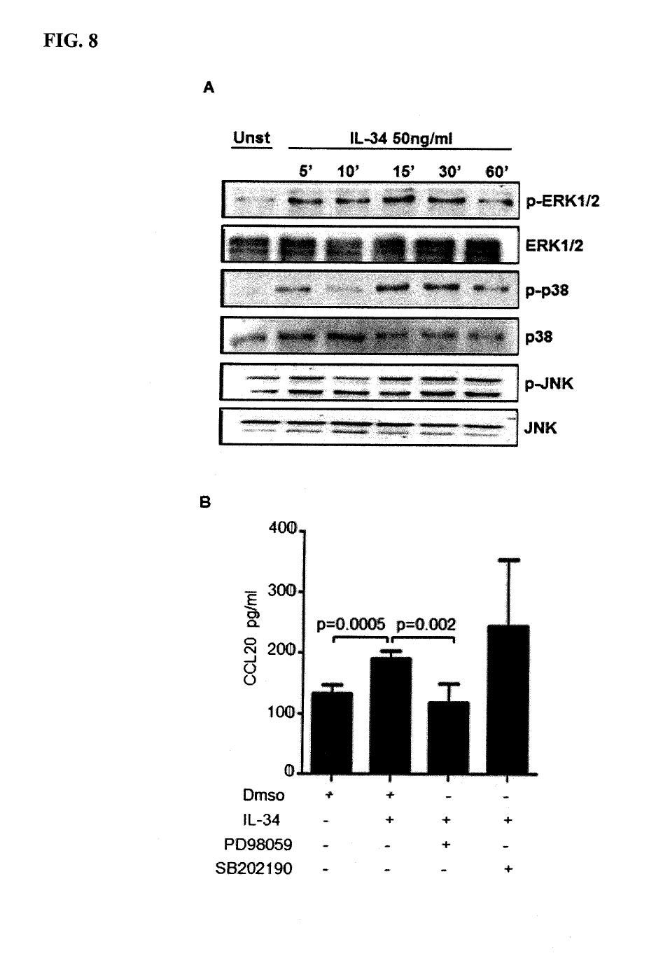

To analyze the role of MAP kinase proteins in facilitating IL-34-stimulated CCL20 expression, activation of the MAP kinases ERK, p38, and JNK by IL-34 was assessed in DLD-1 cells. Serum-starved DLD-1 cells were either left untreated (Unst) or treated with IL-34 at 50 ng/ml for between 5 minutes and 60 minutes. Phosphorylated MAP kinase (p-ERK1/2, p-p38, p-JNK) protein expression representing the activated protein fraction and total MAP kinase (ERK1/2, p38, JNK) protein expression in cell extracts was evaluated by Western blotting in 3 separate experiments (FIG. 8A, showing a representative example). IL-34 rapidly enhanced phosphorylation of both ERK1/2 and p38 but did not noticeably stimulate JNK phosphorylation (FIG. 8A). These results demonstrate that IL-34 exposure stimulated phosphorylation of the ERK1/2 and p38 MAP kinases in colonic epithelial cells.

The ability of individual MAP kinases to facilitate CCL20 production in response to IL-34 stimulation was also evaluated. Serum-starved DLD-1 cells were pre-incubated with a specific inhibitor of ERK1/2 (PD98059) or p38 (SB202190) or with dimethyl sulfoxide (DMSO, vehicle) for 1 hour and left unstimulated or stimulated with IL-34 at 50 ng/mL for an additional 48 hours. CCL20 protein levels were analyzed in cell-free supernatants by ELISA. Pre-incubation of DLD-1 cells with the ERK1/2 inhibitor PD98059, but not with the p38 inhibitor SB202190, abrogated IL-34-induced CCL20 production (FIG. 8B). These result indicates that while IL-34 stimulation activated both p38 and ERK1/2 in colonic epithelial cells, IL-34 stimulated expression of CCL20 was specifically mediated by ERK1/2 activation.

### INCORPORATION BY REFERENCE

The entire disclosure of each of the patent documents and scientific articles cited herein is incorporated by reference for all purposes.

### EQUIVALENTS

The invention can be embodied in other specific forms with departing from the essential characteristics thereof. The foregoing embodiments therefore are to be considered illustrative rather than limiting on the invention described herein. The scope of the invention is indicated by the appended claims rather than by the foregoing description, and all changes that come within the meaning and range of equivalency of the claims are intended to be embraced therein.

1-3. (canceled)

4. A method of treating an inflammatory bowel disease, the method comprising administering to a patient in need thereof an effective amount of an inhibitor of IL-34.

5. A method of treating an inflammatory bowel disease, the method comprising administering a pharmaceutically acceptable formulation comprising an effective amount of an inhibitor of IL-34.

6. The method of claim 5, wherein the administering of said pharmaceutically acceptable formulation comprising an effective amount of an inhibitor of IL-34 is to a patient in need thereof.

7. A method of inhibiting inflammatory cytokine production in cells of a patient suffering from an inflammatory bowel disease, the method comprising administering an effective amount of an inhibitor of IL-34.

8-9. (canceled)

10. The method of claim 4, wherein the inhibitor of IL-34 is an inhibitor of a receptor of IL-34.

11. The method of claim 10, wherein the receptor of IL-34 is Colony Stimulating Factor 1 Receptor.

12. The method of claim 4, wherein the inhibitor of IL-34 comprises an antisense oligonucleotide against IL-34.

13. (canceled)

14. The method of claim 4, wherein the inhibitor of IL-34 is an antibody.

15. (canceled)

16. The method of claim 14, wherein the antibody is an antibody directed against an IL-34 protein sequence of SEQ ID NO: ID: 2 or a Colony Stimulating Factor 1 Receptor protein sequence of SEQ ID NO: 3.

17. The method of claim 4, wherein the inhibitor of IL-34 is a peptide inhibitor.

18. The method of claim 17, wherein the peptide inhibitor comprises a portion of an extracellular domain protein sequence of Colony Stimulating Factor 1 Receptor (SEQ ID NO: 4).

19. The method of claim 4, wherein the administering of the inhibitor of IL-34 is performed topically, parenterally, orally, pulmonarily, intratracheally, intranasally, transdermally, or intraduodenally.

20. The method of claim 4, wherein the effective amount of the inhibitor of IL-34 is administered in the form of a pharmaceutical composition comprising an effective amount of the inhibitor of IL-34 and a pharmaceutically acceptable carrier.

21. The method of claim 20, wherein the administering is performed topically, parenterally, orally, pulmonarily, intratracheally, intranasally, transdermally, or intraduodenally.

22-23 (canceled)

24. A method of diagnosing a patient with an inflammatory bowel disease, the method comprising detecting levels of IL-34 expression signal in one or more biological samples of a patient.

25. A method of determining whether a patient suffering from inflammatory bowel disease is a candidate for treatment with an inhibitor of IL-34, the method comprising detecting levels of IL-34 expression signal in one or more biological samples of said patient.

26-32. (canceled)

33. The method of claim 4, wherein the patient is a human.

34. The method of claim 4, wherein the inflammatory bowel disease is selected from the group consisting of Crohn's disease, gastroduodenal Crohn's disease, Crohn's (granulomatous) colitis, ulcerative colitis, collagenous colitis, lymphocytic colitis, ischaemic colitis, diversion colitis, Behçet's disease, microscopic colitis, ulcerative proctitis, proctosigmoiditis, jejunoileitis, left-sided colitis, pancolitis, ileocolitis, ileitis, and indeterminate colitis.

35-36.(canceled)
